(19) United States
(12) Patent Application Publication
(10) Pub. No.: US 2024/0139189 A1
(43) Pub. Date: May 2, 2024

(54) N6-ADENOSINE-METHYLTRANSFERASE INHIBITORS IN CANCER TREATMENT

(71) Applicant: University of Zurich, Zürich (CH)

(72) Inventors: Amedeo CAFLISCH, Zürich (CH); Aymeric DOLBOIS, Zürich (CH); Danzhi HUANG, Zürich (CH); Frantisek ZALESAK, Zürich (CH)

(21) Appl. No.: 18/506,287

(22) Filed: Nov. 10, 2023

Related U.S. Application Data
(63) Continuation of application No. PCT/EP2022/063350, filed on May 17, 2022.

(30) Foreign Application Priority Data
May 17, 2021 (EP) EP21174041.0
Nov. 30, 2021 (EP) EP21211529.9

(51) Int. Cl. A61K 31/506; A61K 31/519; A61K 31/5386

(52) U.S. Cl. CPC A61K 31/506; A61K 31/519; A61K 31/5386

(57) ABSTRACT

The present invention relates to N6-adenosine-methyltransferase inhibitors and to dual N6-adenosine-methyltransferase E3 ligase binders in cancer treatment.

This application is a continuation of International Application No. PCT/EP2022/063350, filed May 17, 2022, which claims priority to European Patent Applications EP21174041.0, filed May 17, 2021, and EP21211529.9 filed Nov. 30, 2021, which are incorporated herein by reference.

The present invention relates to N6-adenosine-methyltransferase inhibitors and to dual N6-adenosine-methyltransferase E3 ligase binders in cancer treatment.

### BACKGROUND OF THE INVENTION

Expression of genes is regulated at the level of the transcriptome (messenger RNA obtained through transcription of the genome) through dynamic levels of mRNA modifications. The conversion of adenosine to N6-methyladenosine (m⁶A) is the most common internal post-transcriptional modification (also called epitranscriptomic modification) in eukaryotic mRNA. This methylation event typically occurs within the DRACH (D=A, G, U; R=A, G; H=A, C, U) consensus sequence motif. The m⁶A level can vary among different tissues, development states or in response to cellular stresses. On the molecular level introduction of the m⁶A affects the structure of RNA and its ability to form protein-RNA interactions, and as a consequence it modulates processing, translation, and stability of the cellular transcripts. As a consequence, m⁶A is implicated in controlling embryonic development processes and stem cell differentiation, regulating the mammalian circadian clock, and modulating stress response, e.g., heat shock.

The dynamic level of m⁶A is regulated by the interplay of erasers and writer proteins. While the m⁶A writer has been known for two decades, the discovery of m⁶A-specific eraser proteins FTO (ALKBH9) and ALKBHS has ultimately demonstrated the reversibility of the modification and its regulatory role. These m⁶A demethylases belong to the dioxygenase AlkB family whose enzymatic reaction depends on Fe(II) and 2-oxoglutaric acid (2 OG). The core writer complex is formed by two methyltransferase-like proteins, METTL3 and METTL14, which rely on additional cofactors for mRNA substrate recruitment, including WTAP and RBM15. The METTL3-METTL14 complex transfers a methyl group from S-adenosylmethionine (SAM) to the adenosine within the consensus sequence of 5′-GGACU-3′. Only METTL3 has an intact SAM-binding site, while METTL14 possesses a degenerate SAM-binding site, which is not functional. The individual depletion of METTL3 or METTL14 reduces the level of m⁶A in HeLa cells. More importantly, deregulation of METTL3 has recently been linked to specific tumors, such as acute myeloid leukemia, hepatocellular carcinoma, and lung adenocarcinoma. Moreover, inhibiting m⁶A modification shows a broad antiviral effect. Therefore, small-molecule modulators of the METTL3-METTL14 writer has potential therapeutic use in cancer and viral infection. Except for the by-product S-adenosyl-L-homocysteine (SAH), there is no inhibitor reported as of today.

Based on the above-mentioned state of the art, the objective of the present invention is to provide means and methods to use the small molecule therapeutic modalities to modulate the levels of m6A modification with the goal of regulating gene expression for cancer therapy.

This objective is attained by the subject-matter of the independent claims of the present specification.

### SUMMARY OF THE INVENTION

A first aspect of the invention relates to a compound of the general formula (A)

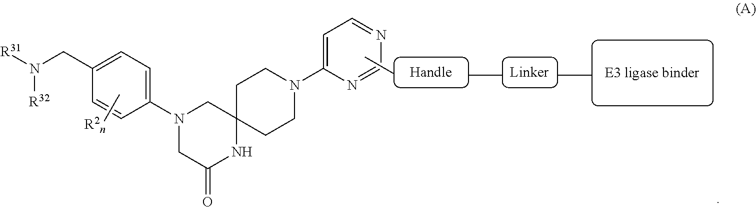

A second aspect of the invention relates to a compound of the general formula (U)

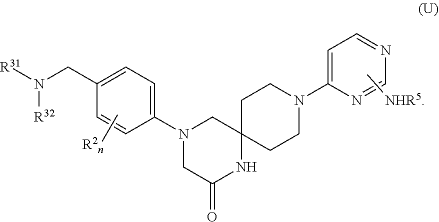

A third aspect of the invention relates to a compound according to the first or second aspect for use as a medicament.

A fourth aspect of the invention relates to a compound according to the first or second aspect for use in treatment of cancer.

In another embodiment, the present invention relates a pharmaceutical composition comprising at least one of the compounds of the present invention or a pharmaceutically acceptable salt thereof and at least one pharmaceutically acceptable carrier, diluent or excipient.

### DETAILED DESCRIPTION OF THE INVENTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term METTL3 in the context of the present specification relates to N6-adenosine-methyltransferase catalytic subunit (Uniprot ID: Q86U44).

The term METTL1 4 in the context of the present specification relates to N6-adenosine-methyltransferase non-catalytic subunit (Uniprot ID: Q9HCE5).

A C₁-C₆ alkyl in the context of the present specification signifies a saturated linear or branched hydrocarbon having 1, 2, 3, 4, 5 or 6 carbon atoms. In certain embodiments, the alkyl is substituted, meaning e.g. one or more CH₂ moieties may be exchanged for oxygen (ether bridge) or nitrogen (NH, or NR with R being methyl, ethyl, or propyl; amino bridge).

The term C₃-C₇ cycloalkyl in the context of the present specification relates to a saturated hydrocarbon ring having 3, 4, 5, 6 or 7 carbon atoms, wherein in certain embodiments, one carbon-carbon bond may be unsaturated. Non-limiting examples of a C₃-C₇ cycloalkyl moiety include cyclopropanyl (—C₃H₅), cyclobutanyl (—C₄-C₇), cyclopentenyl (C₅ H₉), and cyclohexenyl (C₆H₁₁) moieties. In certain embodiments, the cycloalkyl is substituted. In certain embodiments, a cycloalkyl is substituted by one C₁ to C₄ unsubstituted alkyl moiety. In certain embodiments, a cycloalkyl is substituted by more than one C₁ to C₄ unsubstituted alkyl moieties.

The term heterocycle in the context of the present specification relates to a cycloalkyl, wherein at least one ring atom is replaced or several ring atoms are replaced by a nitrogen, oxygen and/or sulphur atom.

The term heterobicycle in the context of the present specification relates to two directly connected cycloalkyls, wherein at least one ring atom is replaced or several ring atoms are replaced by a nitrogen, oxygen and/or sulphur atom.

The term heterocycloalkyl in the context of the present specification relates to a cycloalkyl, wherein at least one ring atom is replaced or several ring atoms are replaced by a nitrogen, oxygen and/or sulphur atom.

The term unsubstituted Cₙ alkyl when used herein in the narrowest sense relates to the moiety if used as a bridge between moieties of the molecule, or if used in the context of a terminal moiety.

The terms unsubstituted Cₙ alkyl and substituted Cₙ alkyl include a linear alkyl comprising or being linked to a cyclical structure, for example a cyclopropane, cyclobutane, cyclopentane or cyclohexane moiety, unsubstituted or substituted depending on the annotation or the context of mention, having linear alkyl substitutions. The total number of carbon and—where appropriate— N, O or other hetero atom in the linear chain or cyclical structure adds up to n.

Where used in the context of chemical formulae, the following abbreviations may be used: Me is methyl CH₃, Et is ethyl-CH₂CH₃, Prop is propyl-(CH₂)₂CH₃ (n-propyl, n-pr) or —CH(CH₃)₂ (iso-propyl, i-pr), but is butyl-C₄-C₉, —(CH₂)₃CH₃, —CHCH₃CH₂CH₃, —CH₂CH(CH₃)₂ or —C(CH₃)₃.

The term substituted alkyl in its broadest sense refers to an alkyl as defined above in the broadest sense, which is covalently linked to an atom that is not carbon or hydrogen, particularly to an atom selected from N, O, F, B, Si, P, S, Cl, Br and I, which itself may be—if applicable— linked to one or several other atoms of this group, or to hydrogen, or to an unsaturated or saturated hydrocarbon (alkyl or aryl in their broadest sense). In a narrower sense, substituted alkyl refers to an alkyl as defined above in the broadest sense that is substituted in one or several carbon atoms by groups selected from amine NH₂, alkylamine NHR, imide NH, alkylimide NR, amino(carboxyalkyl) NHCOR or NRCOR, hydroxyl OH, oxyalkyl OR, oxy(carboxyalkyl) OCOR, carbonyl O and its ketal or acetal (OR)₂, nitril CN, isonitril NC, cyanate CNO, isocyanate NCO, thiocyanate CNS, isothiocyanate NCS, fluoride F, choride Cl, bromide Br, iodide I, phosphonate PO₃H₂, PO₃R₂, phosphate OPO₃H₂ and OPO₃R₂, sulfhydryl SH, suflalkyl SR, sulfoxide SOR, sulfonyl SO₂R, sulfanylamide SO₂NHR, sulfate SO₃H and sulfate ester SO₃R with R being defined further in the description..

The term hydroxyl substituted group refers to a group that is modified by one or several hydroxyl groups OH.

The term amino substituted group refers to a group that is modified by one or several amino groups NH₂.

The term carboxyl substituted group refers to a group that is modified by one or several carboxyl groups COOH.

Non-limiting examples of amino-substituted alkyl include —CH₂NH₂, —CH₂NHMe, —CH₂NHEt, —CH₂CH₂NH₂, —CH₂CH₂NHMe, —CH₂CH₂NHEt, —(CH₂)₃NH₂, —(CH₂)₃NHMe, —(CH₂)₃NHEt, —CH₂CH(NH₂)CH₃, —CH₂CH(NHMe)CH₃, —CH₂CH(NHEt)CH₃, —(CH₂)₃CH₂NH₂, —(CH₂)₃CH₂NHMe, —(CH₂)₃CH₂NHEt, —CH(CH₂NH₂)CH₂CH₃, —CH(CH₂NHMe)CH₂CH₃, —CH(CH₂NHEt)CH₂CH₃, —CH₂CH(CH₂NH₂)CH₃, —CH₂CH(CH₂NHMe)CH₃, —CH₂CH(CH₂NHEt)CH₃, —CH(NH₂)(CH₂)₂NH₂, —CH(NHMe)(CH₂)₂NHMe, —CH(NHEt)(CH₂)₂NHEt, —CH₂CH(NH₂)CH₂NH₂, —CH₂CH(NHMe)CH₂NHMe, —CH₂CH(NHEt)CH₂NHEt, —CH₂CH(NH₂)(CH₂)₂NH₂, —CH₂CH(NHMe)(CH₂)₂NHMe, —CH₂CH(NHEt)(CH₂)₂NHEt, —CH₂CH(CH₂NH₂)₂, —CH₂CH(CH₂NHMe)₂and —CH₂CH(CH₂NHEt)₂ for terminal moieties and —CH₂CHNH₂—, —CH₂CHNHMe—, —CH₂CHNHEt— for an amino substituted alkyl moiety bridging two other moieties.

Non-limiting examples of hydroxy-substituted alkyl include —CH₂OH, —(CH₂)₂OH, —(CH₂)₃OH, —CH₂CH(OH)CH₃, —(CH₂)₄OH, —CH(CH₂OH)CH₂CH₃, —CH₂CH(CH₂OH)CH₃, —CH(OH)(CH₂)₂OH, —CH₂CH(OH)CH₂OH, —CH₂CH(OH)(CH₂)₂OH and —CH₂CH(CH₂OH)₂for terminal moieties and —CHOH—, —CH₂CHOH—, —CH₂CH(OH)CH₂—, —(CH₂)₂CHOHCH₂—, —CH(CH₂OH)CH₂CH₂—, —CH₂CH(CH₂OH)CH₂—, —CH(OH)(CH₂CHOH—, —CH₂CH(OH)CH₂OH, —CH₂CH(OH)(CH₂)₂OH and —CH₂CHCH₂OHCHOH— for a hydroxyl substituted alkyl moiety bridging two other moieties.

The term sulfoxyl substituted group refers to a group that is modified by one or several sulfoxyl groups —SO₂R, or derivatives thereof, with R being defined further in the description.

The term sulfonamide substituted group refers to a group that is modified by one or several sulfonamide groups —SO₂NHR or —NHSO₂R, or derivatives thereof, with R being defined further in the description.

The term amine substituted group refers to a group that is modified by one or several amine groups —NHR or —NR₂, or derivatives thereof, with R being defined further in the description.

The term carbonyl substituted group refers to a group that is modified by one or several carbonyl groups —COR, or derivatives thereof, with R being defined further in the description.

An ester refers to a group of —CO₂R, with R being defined further in the description. An ether refers to a group having one oxygen in between two saturated carbon atoms.

An amide refers to a group of —CONHR, with R being defined further in the description.

An ethylene glycol refers to a group of —(CH₂—CH₂—O)ₙ— or —(O—CH₂—CH₂)ₙ—, with n being defined further in the description.

An alkylyne refers to a group of —C≡C— (triple bond between two carbon atoms).

An alkylene refers to a group of —CH═CH— (double bond between two carbon atoms).

The term halogen-substituted group refers to a group that is modified by one or several halogen atoms selected (independently) from F, Cl, Br, I.

The term fluoro substituted alkyl refers to an alkyl according to the above definition that is modified by one or several fluoride groups F. Non-limiting examples of fluoro-substituted alkyl include —CH₂F, —CHF₂, —CF₃, —(CH₂)₂F, —(CHF)₂H, —(CHF)₂F, —C₂F₅, —(CH₂)₃F, —(CHF)₃H, —(CHF)₃F, —C₃F₇, —(CH₂)₄F, —(CHF)₄H, —(CHF)₄F and —C₄F₉.

Non-limiting examples of hydroxyl- and fluoro-substituted alkyl include —CHFCH₂OH, —CF₂CH₂OH, —(CHF)₂CH₂OH, —(CF₂)₂CH₂OH, —(CHF)₃CH₂OH, —(CF₂)₃CH₂OH, —(CH₂)₃OH, —CF₂CH(OH)CH₃, —CF₂CH(OH)CF₃, —CF(CH₂OH)CHFCH₃, and —CF(CH₂OH)CHFCF₃.

The term aryl in the context of the present specification signifies a cyclic aromatic C₅-C₁₀ hydrocarbon. Examples of aryl include, without being restricted to, phenyl and naphthyl.

An alkylaryl in the context of the present specification relates to an alkyl group substituted by an aryl moiety. Particular examples are ethylphenyl, propylphenyl, butylphenyl and their higher homologues. A substituted alkyl aryl may be substituted by the substituent indicated on the alkyl part, if chemically feasible, or on the aryl part of the moiety.

A heteroaryl is an aryl that comprises one or several nitrogen, oxygen and/or sulphur atoms. Examples for heteroaryl include, without being restricted to, pyrrole, thiophene, furan, imidazole, pyrazole, thiazole, oxazole, pyridine, pyrimidine, thiazin, quinoline, benzofuran and indole. A heteroaryl also encompasses a bicyclic heteroaryl. An aryl or a heteroaryl in the context of the specification additionally may be substituted by one or more alkyl groups.

An alkylheteroaryl in the context of the present specification relates to an alkyl group substituted by a heteroaryl moiety.

As used herein, the term pharmaceutical composition refers to a compound of the invention, or a pharmaceutically acceptable salt thereof, together with at least one pharmaceutically acceptable carrier. In certain embodiments, the pharmaceutical composition according to the invention is provided in a form suitable for topical, parenteral or injectable administration.

As used herein, the term pharmaceutically acceptable carrier includes any solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington: the Science and Practice of Pharmacy, ISBN 0857110624)

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease

### PROTAC Compound

A first aspect of the invention relates to a compound of the general formula (A)

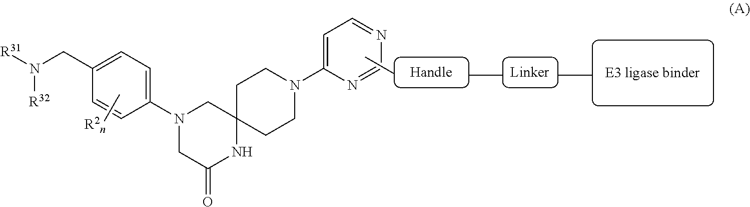

wherein
   NR³¹R³² is selected from

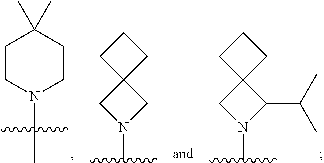

each R² is independently selected from the group comprising F, Cl, CF₃, CHF₂, CH₂F;
   n is an integer selected from 0, 1, 2, 3, and 4;
   Handle is a connecting moiety comprising or essentially consisting of 3 to 10 atoms of atomic mass (C, N, O, S);
   Linker is a linker moiety comprising or essentially consisting of 3 to 50 atoms of atomic mass≥12;
   E3 ligase binder is a moiety specifically binding to an E3 ligase.

In certain embodiments, each R² is F. In certain embodiments, n is an integer selected from 0, 1, and 2. In certain embodiments, n is 2. In certain embodiments, Handle is a connecting moiety comprising or essentially consisting of 4 to 8 atoms of atomic mass≥12. In certain embodiments, Linker is a linker moiety comprising or essentially consisting of 4 to 30 atoms of atomic mass≥12. In certain embodiments, Linker is a linker moiety comprising or essentially consisting of 5 to 20 atoms of atomic mass≥12.

### E3 Ligase Binder

An E3 ligase binder is a molecule which specifically binds an E3 ligase. In certain embodiments, the E3 ligase is cereblon (UniProt-ID: Q96SW2).

In certain embodiments, the E3 ligase binder is of the formula (B)

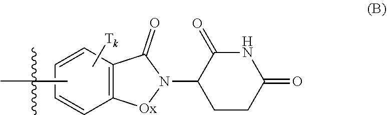

wherein
   Ox is CH₂ or C═O;
   T is selected from the group comprising F, Cl;
   k is an integer selected from the group comprising 0, 1, 2;
   [symbol] designates the bond to the Linker.

In certain embodiments, k is an integer selected from the group comprising 0, 1. In certain embodiments, k is 0. In certain embodiments, T is F.

### Handle

In certain embodiments, Handle is a connecting moiety comprising or essentially consisting of 3 to 10 atoms of atomic mass (C, N, O, S). In certain embodiments, Handle is a connecting moiety comprising or essentially consisting of 4 to 8 atoms of atomic mass≥12.

In certain embodiments, the Handle comprises or essentially consists of 1, 2, 3, or 4 chemical moieties selected from the group comprising alkyl, amine, phenyl, and carbonyl.

In certain embodiments, the Handle is selected from the group comprising the following formulas:

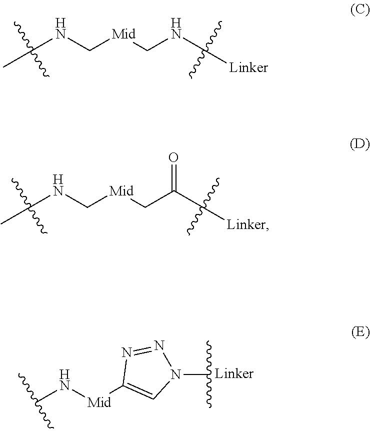

wherein
   Mid is selected from the group comprising C₁-C₃ alkyl, and phenyl.

In certain embodiments, the Handle is selected from the group comprising the following formulas:

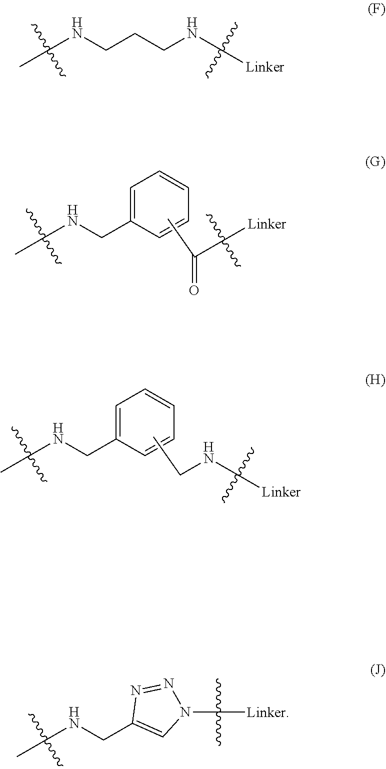

### Linker

In certain embodiments, Linker is a linker moiety comprising or essentially consisting of 3 to 50 atoms of atomic mass (C, N, O, S). In certain embodiments, Linker is a linker moiety comprising or essentially consisting of 4 to 30 atoms of atomic mass≥12. In certain embodiments, Linker is a linker moiety comprising or essentially consisting of 5 to 20 atoms of atomic mass≥12.

In certain embodiments, the Linker comprises or essentially consists of 1, 2, 3, 4, 5, 6, or 7 chemical moieties independently selected from the group comprising alkyl, aryl, heteroaryl, cycloalkyl, heterocycloalkyl, alkylene, alkylyne, ethylene glycol, carbonyl, ether, ester, amine, amide, sulfonamide, wherein the chemical moieties are each independently unsubstituted or substituted with C₁-C₃ alkyl, halogen, CN, NO₂, hydroxyl, amine, sulfate, phosphate, and/or carboxyl.

In certain embodiments, the Linker comprises or essentially consists of 1, 2, 3, or 4 chemical moieties selected from the group comprising alkyl, ethylene glycol, carbonyl, piperazine, aryl, amine, triazole.

In certain embodiments, the Linker is selected from the group comprising the following formulas:

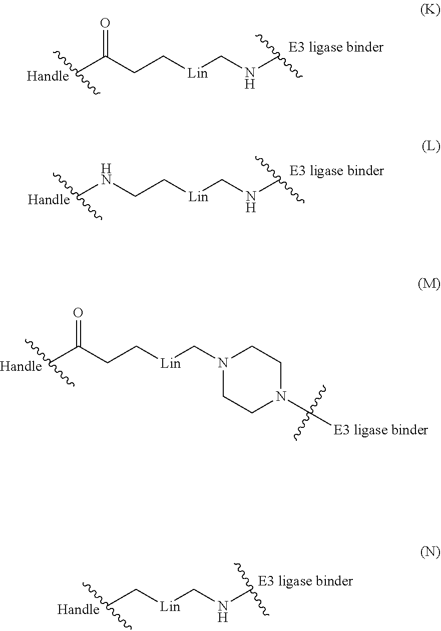

wherein
   Lin is selected from the group comprising C₃-C₂₀ alkyl, C₃-C₂₀ alkyl-triazole, oligo(ethylene glycol).

In certain embodiments, the Linker is selected from the group comprising the following formulas:

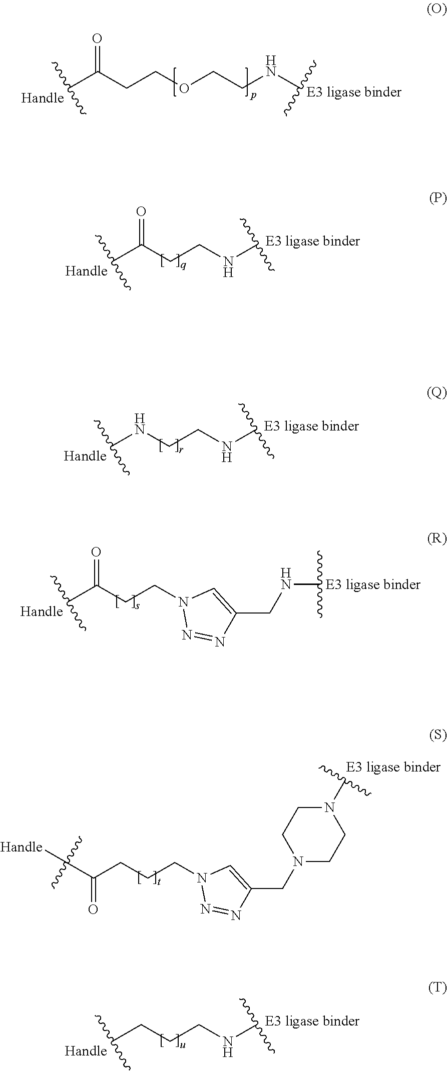

wherein
   p is selected from 2, 3, 4, 5;
   q is selected from 7, 8, 9, 10, 11, 12, 13;
   r is selected from 11, 12, 13, 14, 15, 16, 17;
   s is selected from 7, 8, 9, 10, 11, 12, 13;
   t is selected from 3, 4, 5, 6, 7, 8, 9;
   u is selected from 7, 8, 9, 10, 11, 12, 13.

In certain embodiments, the Linker is a peptide. In certain embodiments, the Linker is a peptide consisting of proteinogenic amino acids.

### Combination of Features

In certain embodiments,
the E3 ligase binder is of the formula (B); and
the Handle is of formula (F), (G), (H), or (J); and
the Linker is of formula (O); (P); (Q); (R); (S); or (T).

In certain embodiments, the compound comprises the following definitions of the Handle, Linker and E3 ligase binder (one row is one combination):

| Handle | Linker | E3 ligase binder |
|---|---|---|
| 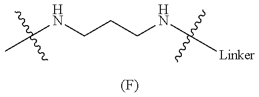 [SMILES: *NCCCN* ; "(F)"] | 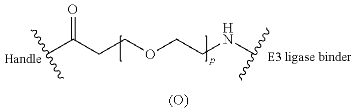 | 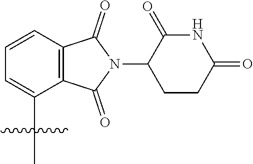 [SMILES: *c1cccc2c1C(=O)N(C1CCC(=O)NC1=O)C2=O] |
| 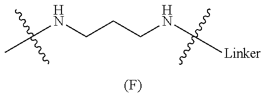 [SMILES: *NCCCN* ; "(F)"] | 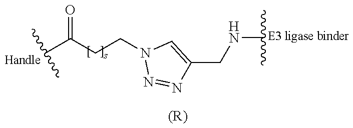 | 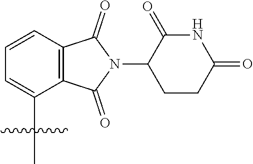 [SMILES: *c1cccc2c1C(=O)N(C1CCC(=O)NC1=O)C2=O] |
| | | 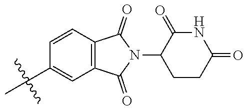 [SMILES: *c1ccc2c(c1)C(=O)N(C1CCC(=O)NC1=O)C2=O] |
| 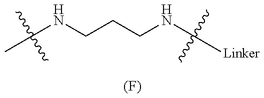 [SMILES: *NCCCN* ; "(F)"] | 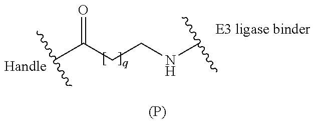 | 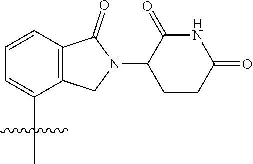 [SMILES: *c1cccc2c1CN(C1CCC(=O)NC1=O)C2=O] |
| | | 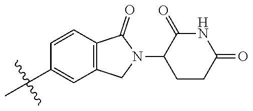 [SMILES: *c1ccc2c(c1)CN(C1CCC(=O)NC1=O)C2=O] |
| 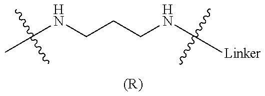 [SMILES: *NCCCN* ; "(R)"] | 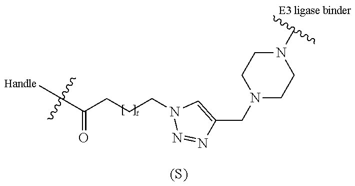 | 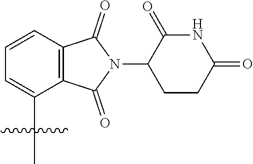 [SMILES: *c1cccc2c1C(=O)N(C1CCC(=O)NC1=O)C2=O] |
| | | 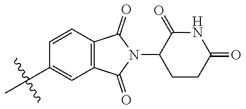 [SMILES: *c1ccc2c(c1)C(=O)N(C1CCC(=O)NC1=O)C2=O] |
| 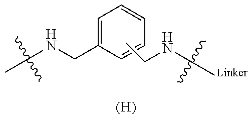 | 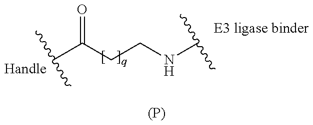 | 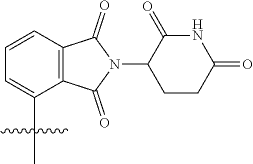 [SMILES: *c1cccc2c1C(=O)N(C1CCC(=O)NC1=O)C2=O] |
| | | 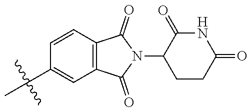 [SMILES: *c1ccc2c(c1)C(=O)N(C1CCC(=O)NC1=O)C2=O] |
| 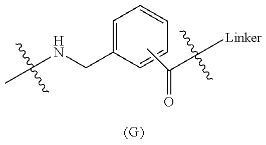 | 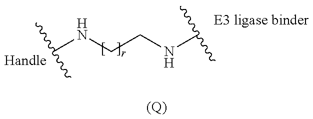 | 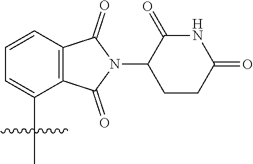 [SMILES: *c1cccc2c1C(=O)N(C1CCC(=O)NC1=O)C2=O] |
| | | 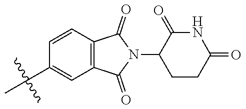 [SMILES: *c1ccc2c(c1)C(=O)N(C1CCC(=O)NC1=O)C2=O] |
| 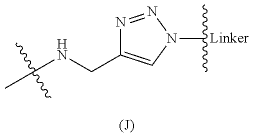 [SMILES: *NCc1cn(*)nn1] | 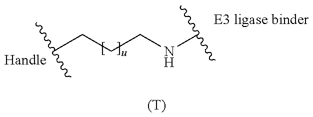 | 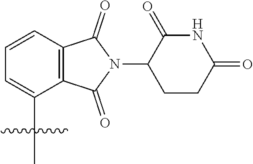 [SMILES: *c1cccc2c1C(=O)N(C1CCC(=O)NC1=O)C2=O] |
| | | 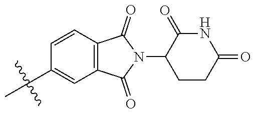 [SMILES: *c1ccc2c(c1)C(=O)N(C1CCC(=O)NC1=O)C2=O] |

### Active Compound

A second aspect of the invention relates to a compound of the general formula (I)

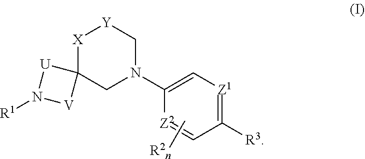

An alternative of the second aspect of the invention relates to a compound of the general formula (Ia)

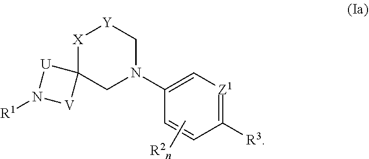

An alternative of the second aspect of the invention relates to a compound of the general formula (Ib)

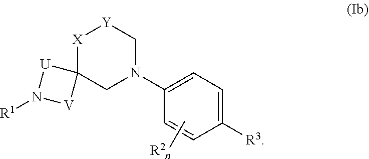

wherein
   Z¹ and Z² are independently selected from N, CH and CR²;
   X is O or NH;
   Y is CH₂, C═O, or SO₂;
   R¹ is an unsubstituted or substituted moiety selected from aryl, heteroaryl, cycloalkyl, and a heterocycle, particularly R¹ is unsubstituted or substituted heteroaryl;
   R² is selected from F, Me, Cl, OH, NH₂, Br, CF₃, CHF₂, CH₂F;
   n is an integer selected from 0, 1, 2, 3, and 4;
   R³ is a substituted alkylamine;
   U and V are independently selected from —CH₂— and —(CH₂)₂—, or one of U and V is —CH₂— and the other one is —(CH₂)₃—.

In certain embodiments, X is NH. In certain embodiments, Y is C═O. In certain embodiments, n is an integer selected from 0, 1, and 2. In certain embodiments, U and V are both —CH₂— or are both —(CH₂)₂—.

In certain embodiments, the compound is of the general formula (U)

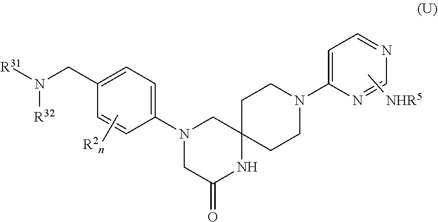

NR³¹R³² is selected from

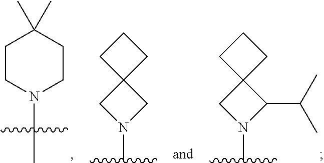

R² is selected from the group comprising F, Cl, CF₃, CHF₂, CH₂F;
   n is an integer selected from 0, 1, 2, 3, and 4;
   R⁵ is selected from an alkyl, an alkylaryl, a heteroalkylaryl, a cycloalkyl, an aryl, a heteroaryl and a heterocycle.

In certain embodiments, R² is F. In certain embodiments, n is an integer selected from 0, 1, and 2. In certain embodiments, n is 2. In certain embodiments, R⁵ is selected from an alkyl, an alkylaryl, and a cycloalkyl. In certain embodiments, R⁵ is selected from methyl and methylphenyl.

### Middle Ring System

In certain embodiments, X is NH. In certain embodiments, Y is C═O.

In certain embodiments, the moiety

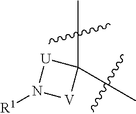

is selected from

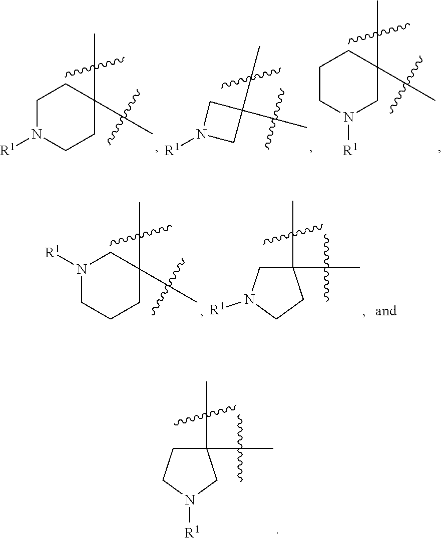

In certain embodiments, the moiety

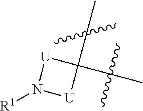

is selected from

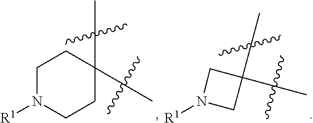

### Moiety R¹

In certain embodiments, R¹ is unsubstituted or substituted heteroaryl. In certain embodiments, R¹ is unsubstituted or substituted with a moiety selected from
a secondary amine NHR^{N}, wherein R^{N} is selected from a C₁-C₆ alkyl, a C₄-C₆ cycloalkyl, an aryl, and a heteroaryl, an alkylaryl, and an alkylheteroaryl;
a halogen, particularly Cl or F;
a C₁-C₆ alkyl, a C₄-C₆ cycloalkyl, an aryl, and a heteroaryl.

In certain embodiments, R¹ is unsubstituted or substituted with a moiety selected from
a secondary amine NHR^{N}, wherein R^{N} is selected from a C₁-C₆ alkyl, a C₄-C₆ cycloalkyl, an aryl, and a heteroaryl;
a halogen, particularly Cl or F.

In certain embodiments, the compound is of the general formula (II)

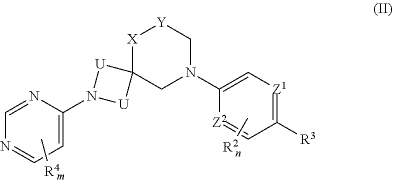

wherein
   Z¹, Z², X, Y, R², R³, U, V, and n have the same definitions as defined above;
   each R⁴ is independently selected from
      a secondary amine substituted with an alkyl, an alkylaryl, a heteroalkylaryl, a cycloalkyl, an aryl, a heteroaryl and/or a heterocycle,
      a halogen;
   and/or two R⁴ together form an unsubstituted or substituted heteroaryl or heterocycle;
   m is an integer selected from 0, 1, 2, and 3.

In certain embodiments, the compound is of the general formula (III)

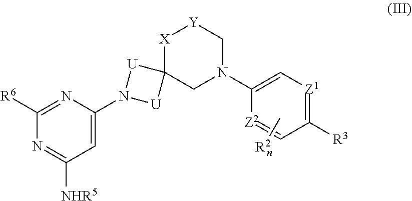

wherein
Z¹, Z², X, Y, R², R³, U, V, and n have the same definitions as defined above;
R⁵ is selected from an alkyl, an alkylaryl, a heteroalkylaryl, a cycloalkyl, an aryl, a heteroaryl and a heterocycle;
R⁶ is selected from halogen and hydrogen.

In certain embodiments, the compound is of the general formula (IV)

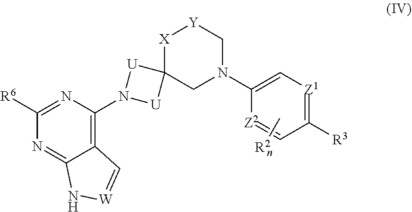

wherein
Z¹, Z², X, Y, R², R³, U, V, and n have the same definitions as defined above;
R⁶ is selected from halogen and hydrogen;
W is selected from N and CH.

In certain embodiments, at least one of Z¹ and Z² is CH or CR². In certain embodiments, both Z¹ and Z² are CH or CR².

### Moiety R³

In certain embodiments, R³ is substituted with one or several moieties selected independently from alkyl-, hydroxy-, amino-, amine-, halogen-, cycloalkyl-, and heterocycle-moieties.

In certain embodiments, R³ is substituted C₁-C₄ alkylamine. In certain embodiments, R³ is substituted C₁-C₂ alkylamine.

In certain embodiments, R³ is

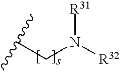

wherein
   s is an integer selected from 1 and 2, more particularly s is 1;
   R³¹ and R³² together form a heterocycle or heterobicycle, which is unsubstituted or alkyl—, halogen—, and/or hydroxyl-substituted, or
   R³¹ and R³² are independently selected from hydrogen and unsubstituted or hydroxy—, and/or halogen-substituted alkyl or cycloalkyl.

In certain embodiments, R³¹ and/or R³² are unsubstituted or substituted with alkyl-, hydroxy-, halogen-, cycloalkyl-, heterocycle- and/or -groups. In certain embodiments, R³¹ and/or R³² are independently selected from H and unsubstituted or hydroxy—, and/or halogen-substituted alkyl and cycloalkyl. In certain embodiments, R³¹ and R³² together form a heterocycle or heterobicycle, which is unsubstituted or alkyl—, hydroxy—, and/or halogen-substituted.

In certain embodiments, NR³¹R³² is selected from

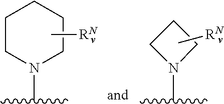

with v being an integer selected from 0, 1 and 2 and each R^{N} being independently selected from hydroxyl, halogen, and C₁-C₄ alkyl, or two R^{N} form a C₃-C₆ cycloalkyl.

In certain embodiments, —NR³¹R³² is selected from

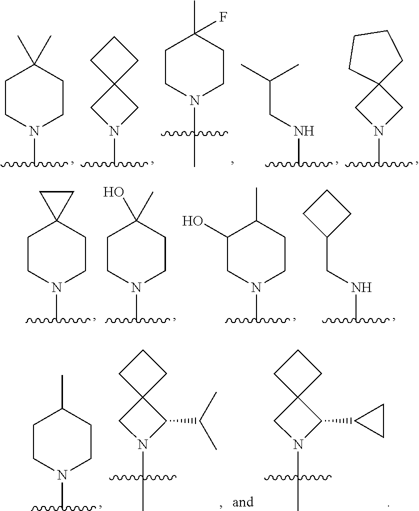

In certain embodiments, NR³¹R³² is selected from

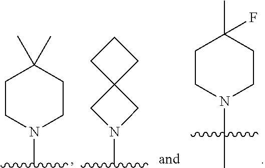

In certain embodiments, NR³¹R³² is

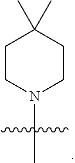 [SMILES: *N1CCC(C)(C)CC1]

In certain embodiments, NR³¹R³² is

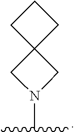 [SMILES: *N1CC2(CCC2)C1]

In certain embodiments, NR³¹R³² is

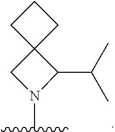 [SMILES: *N1CC2(CCC2)C1C(C)C]

In certain embodiments, NR³¹R³² is

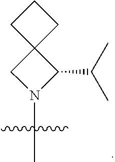 [SMILES: *N1CC2(CCC2)[C@@H]1C(C)C]

### Moiety R²

In certain embodiments, n is an integer selected from 0, 1, and 2. In certain embodiments, n is 2. In certain embodiments, R² is selected from F, Cl and OH. In certain embodiments, R² is F. R² can be bound to any of the carbon atoms of the aryl-or heteroaryl-ring. Thus, it can also be bound to Z¹ or Z² if they are carbon atoms.

### Moiety R⁵

In certain embodiments, R⁵ is selected from an alkyl, an alkylaryl, and a cycloalkyl. In certain embodiments, R⁵ is selected from methyl and methylphenyl.

### Use of the Compound

A third aspect of the invention relates to a compound according to the first or second aspect for use as a medicament.

A fourth aspect of the invention relates to a compound according to the first or second aspect for use in treatment of cancer.

In certain embodiments, the cancer is selected from the group comprising renal cancer, breast cancer, acute myeloid leukemia, hepatocellular carcinoma, and lung adenocarcinoma.

Similarly, within the scope of the present invention is a method or treating cancer in a patient in need thereof, comprising administering to the patient a compound according to the above description.

Similarly, a dosage form for the prevention or treatment of cancer is provided, comprising a non-agonist ligand or antisense molecule according to any of the above aspects or embodiments of the invention.

The skilled person is aware that any specifically mentioned drug may be present as a pharmaceutically acceptable salt of said drug. Pharmaceutically acceptable salts comprise the ionized drug and an oppositely charged counterion. Non-limiting examples of pharmaceutically acceptable anionic salt forms include acetate, benzoate, besylate, bitatrate, bromide, carbonate, chloride, citrate, edetate, edisylate, embonate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate, phosphate, diphosphate, salicylate, disalicylate, stearate, succinate, sulfate, tartrate, tosylate, triethiodide and valerate. Non-limiting examples of pharmaceutically acceptable cationic salt forms include aluminium, benzathine, calcium, ethylene diamine, lysine, magnesium, meglumine, potassium, procaine, sodium, tromethamine and zinc.

Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

### Pharmaceutical Composition and Administration

Another aspect of the invention relates to a pharmaceutical composition comprising a compound of the present invention, or a pharmaceutically acceptable salt thereof, and a pharmaceutically acceptable carrier. In further embodiments, the composition comprises at least two pharmaceutically acceptable carriers, such as those described herein.

In certain embodiments of the invention, the compound of the present invention is typically formulated into pharmaceutical dosage forms to provide an easily controllable dosage of the drug and to give the patient an elegant and easily handleable product.

The pharmaceutical composition can be formulated for oral administration, parenteral administration, or rectal administration. In addition, the pharmaceutical compositions of the present invention can be made up in a solid form (including without limitation capsules, tablets, pills, granules, powders or suppositories), or in a liquid form (including without limitation solutions, suspensions or emulsions).

The dosage regimen for the compounds of the present invention will vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In certain embodiments, the compounds of the invention may be administered in a single daily dose, or the total daily dosage may be administered in divided doses of two, three, or four times daily.

In certain embodiments, the pharmaceutical composition or combination of the present invention can be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The pharmaceutical compositions of the present invention can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc. They may be produced by standard processes, for instance by conventional mixing, granulating, dissolving or lyophilizing processes. Many such procedures and methods for preparing pharmaceutical compositions are known in the art, see for example L. Lachman et al. The Theory and Practice of Industrial Pharmacy, 4th Ed, 2013 (ISBN 8123922892).

### Method of Manufacture and Method of Treatment According to the Invention

The invention further encompasses, as an additional aspect, the use of a compound as identified herein, or its pharmaceutically acceptable salt, as specified in detail above, for use in a method of manufacture of a medicament for the treatment or prevention of cancer.

Similarly, the invention encompasses methods of treatment of a patient having been diagnosed with a disease associated with cancer. This method entails administering to the patient an effective amount of a compound as identified herein, or its pharmaceutically acceptable salt, as specified in detail herein.

Wherever alternatives for single separable features such as, for example, a ligand type or medical indication, are laid out herein as “embodiments”, it is to be understood that such alternatives may be combined freely to form discrete embodiments of the invention disclosed herein. Thus, any of the alternative embodiments for a ligand type may be combined with any medical indication mentioned herein.

The application further encompasses the following items:

### Items

1. A compound of the general formula (I)

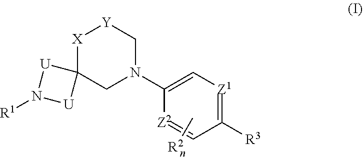

Z¹ and Z² are independently selected from N, CH and CR²;
   X is O or NH, particularly X is NH;
   Y is CH₂, C═O, or SO₂, particularly Y is C═O;
   R¹ is an unsubstituted or substituted moiety selected from aryl, heteroaryl, cycloalkyl, and a heterocycle, particularly R¹ is unsubstituted or substituted heteroaryl;
   R² is selected from F, Me, Cl, OH, NH₂, Br, CF₃, CHF₂, CH₂ F;
   n is an integer selected from 0, 1, 2, 3, and 4, particularly n is an integer selected from 0, 1, and 2;
   R³ is a substituted alkylamine;
   U and V are independently selected from —CH₂— and —(CH₂)₂—, or one of U and V is —CH₂— and the other one is —(CH₂)₃—, particularly U and V are both —CH₂— or are both —(CH₂)₂—.
2. The compound according to item 1, wherein
   R¹ is unsubstituted or substituted with a moiety selected from
      a secondary amine NHR^{N}, wherein R^{N} is selected from a C₁-C₆ alkyl, a C₄-C₆ cycloalkyl, an aryl, and a heteroaryl, an alkylaryl, and an alkylheteroaryl;
      a halogen, particularly Cl or F; and
      a C₁-C₆ alkyl, a C₄-C₆ cycloalkyl, an aryl, and a heteroaryl.
3. The compound according to any one of the preceding items, wherein
   R³ is substituted with one or several moieties selected independently from alkyl—, hydroxy—, amino—, amine—, halogen—, cycloalkyl—, and heterocycle-moieties.
4. The compound according to any one of the preceding items, wherein R³ is C₁-C₄ alkylamine, particularly R³ is C₁-C₂ alkylamine.
5. The compound according to any one of the preceding items, wherein the compound is of the general formula (II)

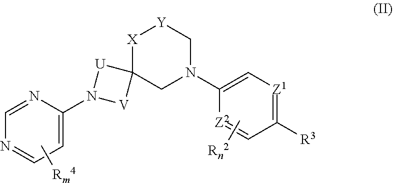

wherein
   Z¹, Z², X, Y, R², R³, U, V, and n have the same definitions as in item 1;
   each R⁴ is independently selected from
      a secondary amine substituted with an alkyl, an alkylaryl, a heteroalkylaryl, a cycloalkyl, an aryl, a heteroaryl and/or a heterocycle, particularly substituted with an alkyl, an alkylaryl, or a cycloalkyl;
      a halogen;
   and/or two R⁴ together form an unsubstituted or substituted heteroaryl or heterocycle;
   m is an integer selected from 0, 1, 2, and 3.
6. The compound according to any one of the preceding items, wherein the compound is of the general formula (Ill)

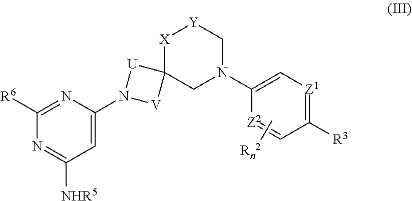

wherein
   Z¹, Z², X, Y, R², R³, U, V, and n have the same definitions as in item 1;
   R⁵ is selected from an alkyl, an alkylaryl, a heteroalkylaryl, a cycloalkyl, an aryl, a heteroaryl and a heterocycle, particularly R⁵ is selected from an alkyl, an alkylaryl, and a cycloalkyl;
   R⁶ is selected from halogen and hydrogen.
7. The compound according to any one of the preceding items 1 to 4, wherein the compound is of the general formula (IV)

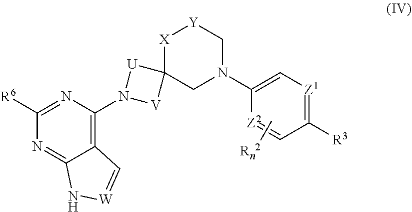

wherein
   Z¹, Z², X, Y, R², R³, U, V, and n have the same definitions as in item 1;
   R⁶ is selected from halogen and hydrogen;
   W is selected from N and CH.
8. The compound according to any one of the preceding items, wherein at least one of Z¹ and Z² is CH or CR², particularly both Z¹ and Z² are CH or CR².
9. The compound according to any one of the preceding items, wherein R³ is

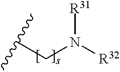

wherein
   is an integer selected from 1 and 2, more particularly s is 1;
   R³¹ and R³² together form a heterocycle or heterobicycle, which is unsubstituted or alkyl—, halogen—, and/or hydroxyl-substituted, or R³¹ and R³² are independently selected from hydrogen and unsubstituted or hydroxy—, and/or halogen-substituted alkyl or cycloalkyl, particularly R³¹ and R³² together form a heterocycle or heterobicycle, which is unsubstituted or alkyl—, halogen—, and/or hydroxyl-substituted.
10. The compound according to item 9, wherein NR³¹R³² is selected from

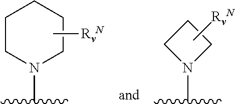

with v being an integer selected from 0, 1 and 2 and each R^{N} being independently selected from hydroxyl, halogen, and C₁-C₄ alkyl, or two R^{N} form a C₃-C₆ cycloalkyl.
11. The compound according to any one of the preceding items 9 to 10, wherein NR³¹R³² is selected from

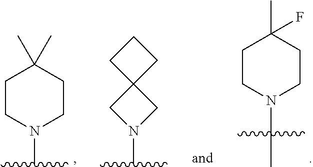

12. The compound according to any one of the preceding items, wherein R² is selected from F, Cl and OH, particularly R² is F.
13. A compound according to any of the preceding items for use as a medicament.
14. A compound according to any of the preceding items 1 to 12 for use in treatment of cancer.

The invention is further illustrated by the following examples and figures, from which further embodiments and advantages can be drawn. These examples are meant to illustrate the invention but not to limit its scope.

### DESCRIPTION OF FIGURES

FIG. 1 shows A) Design of METTL3 inhibitor 2 from hit compound 1. The bonds formed are depicted in red. The IC₅₀ values refer to the biochemical assay based on time resolved-Forster resonance energy transfer (TR-FRET). B) Superimposition of compounds 1 (grey, from X-ray with METTL3, PDB code: 7NHI) and 2 (cyan, drawn in Pymol).

FIG. 2 shows design of compound 8 starting from 5, the newly formed bonds are depicted in red. A) Inhibitor 5 (cyan) in the METTL3 binding site with relevant residues (carbon atoms in grey). The main intermolecular interactions are displayed (yellow dashed lines, PDB code: 7O08). B) Superimposition of inhibitors 5 (cyan) and 7 (green, PDB code: 7O09). C). Superimposition of inhibitors 5 (cyan) and 8 (yellow, PDB code: 7O0L), and interactions of the lactam with the side chain amide of GIn550.

FIG. 3 shows A) Unusual interaction of the fluorine atom of compound 20 with Pro397 amide π-system, PDB code: 7O29. B) Van der Waals contacts between the fluorine atom of inhibitor 21 and the side chains of Ser511 and Tyr406, PDB code: 7O2E.

FIG. 4 shows TR-FRET dose response curves (n≥3) measured for compounds 1, 22 and SAH and chemical structure of the lead compound 22. B) InCELL Pulse dose response curve (n=3) in HEK293T cells at 46° C. C) Representative western blot image for CETSA at 54° C. in MOLM-13 cells and the quantification curve. D) Dose response curves of m6A/A reduction in polyadenylated RNA fraction in MOLM-13 (n=5) and PC-3 (n=3) cell lines measured by UPLC-MS/MS.

FIG. 5 shows thermal shift assay results. Shown are the first derivative of the melting curves of METTL3/METTL14 for inhibitor 22 or SAH.

FIG. 6 shows thermal shift assay results. Shown are the first derivative of the melting curves of METTL1 for inhibitor 22 r SAH. Compound >does not shift the melting temperature of METTL1.

FIG. 7 Exemplary compounds.

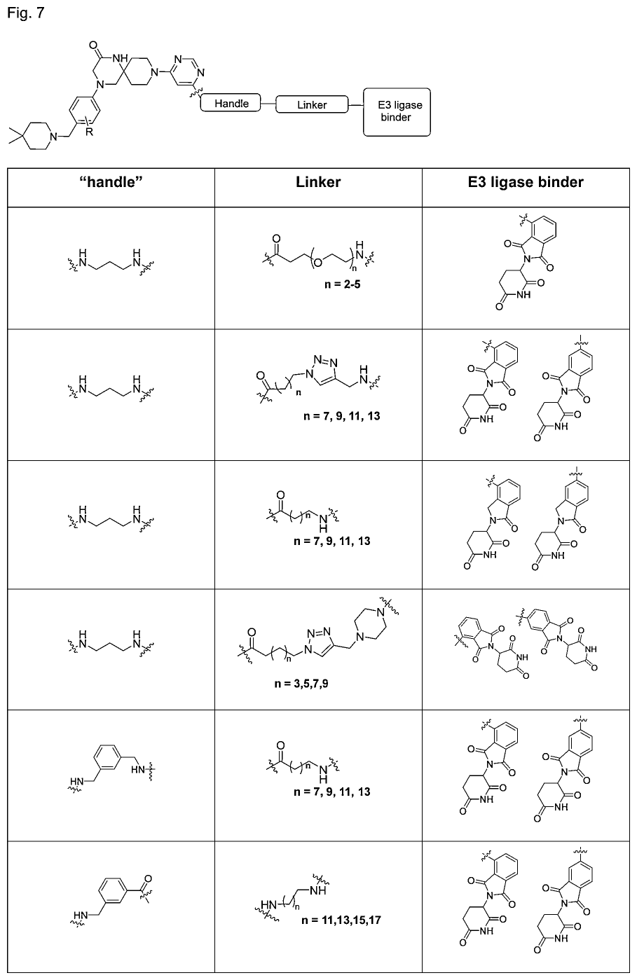

FIG. 8 Exemplary substitution patterns for R.

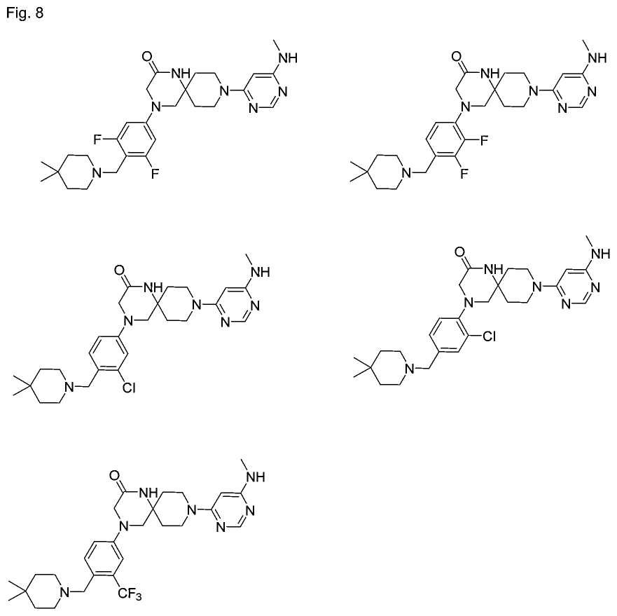

### EXAMPLES

### General Procedure for Buchwald-Hartwig Coupling:

To a stirred solution of the corresponding halide (1 equiv.) in dioxane (0.3 M), under a nitrogen atmosphere, the corresponding amine (1 equiv.) was added. Nitrogen gas was bubbled through the reaction for two minutes and Cs₂ CO₃ (1.2 equiv), Ruphos Pd G4 (10 mol %) and Ruphos (10 mol %) were added. The reaction mixture was stirred at 150° C. for 17 h, concentrated under reduced pressure and the obtained residue was purified by flash column chromatography.

### General Procedure for Boc Group Deprotection:

To a stirred solution of the corresponding Boc protected amine in MeOH (0.3 M), HCl (0.9 M, 37% aq.) was added. The reaction mixture was stirred at 25° C. for 4 h and the reaction mixture was concentrated under reduced pressure. The obtained residue was directly engaged in the next step without further purification.

General Procedure for S_{N}Ar with 4,6-dichloropyrimidine:

To a stirred solution of the corresponding amine (1 eq.) or amine hydrochloride salt (1 equiv.) in iPrOH (0.3 M), 4,6-dichloro-pyrimidine (1.2 equiv.) and Et₃N (1-4 equiv.) were added. The reaction mixture was stirred at 80° C. for 3 h in the microwave and concentrated under reduced pressure. The crude residue was dissolved in nBuOH, washed three times with water, once with brine, dried over MgSO₄ and concentrated under reduced pressure. The crude residue was coevaporated with toluene several times to remove the residual nBuOH and then purified by flash column chromatography.

General Procedure for S_{N}Ar with Chloropyrimidine Derivatives:

The corresponding chloropyrimidine (1 eq.) was dissolved in methylamine (0.1 M, 8 M in EtOH) or benzylamine (0.3 M) and the reaction mixture was stirred at 130° C. for 3 h (MeNH₂) or 140° C. for 8 h (BnNH₂) in the microwave. The crude residue was concentrated under reduced pressure and purified by flash column chromatography. For reactions with benzylamine, the crude residue was coevaporated with water then toluene several times to remove the benzylamine before performing the purification.

### Example 1

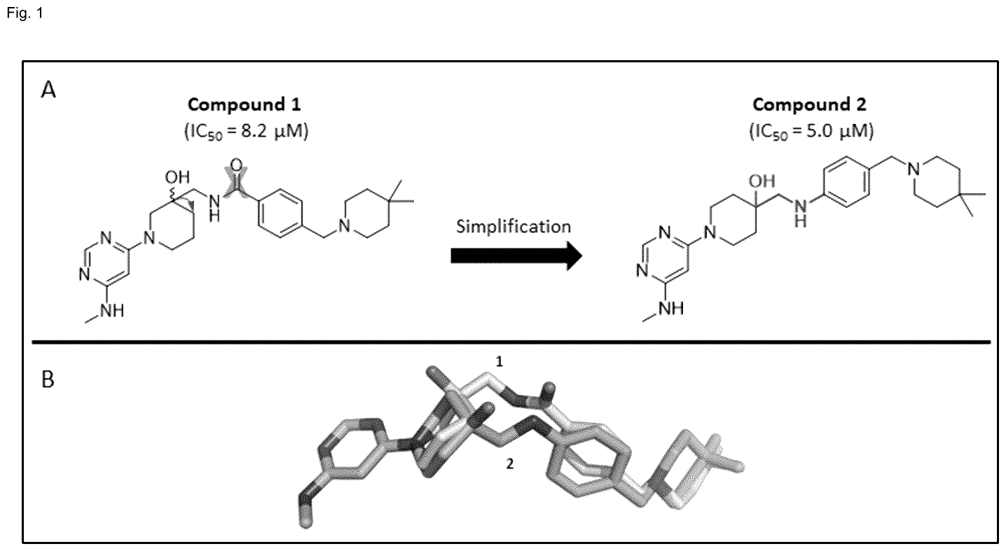

The inventors' design started at the roots of one of the inventor's early inhibitor (1, Table 1), with the aim to simplify the structure and reduce molecular weight (FIG. 1A). For this purpose, changing the methylene position from 1,3 to 1,4 on the piperidine ring removes the chiral center. In addition, according to the X-ray structure of 1 with METTL3, the amide C═O group deletion would allow to keep the original vector (FIG. 1B). These two modifications led to 2 and its two pyridine containing derivatives 3 and 4, which exhibited not only similar potency compared to the parent molecule (IC₅₀=5.0, 4.6, and 5.8 μM respectively, Table 1), but also no chirality and a reduced heavy atom count, hence higher ligand efficiency (LE=0.23, 0.23, and 0.22, respectively). Because 4 had better lipophilic ligand efficiency (LLE=3.4, calculated with DataWarrior), its pyridine core was conserved in the next optimization stage. According to the crystal structure of the complex of METTL3 with inhibitor 1, methylamine to benzylamine replacement on the pyrimidine ring seemed beneficial to inhibition. This proved to be true as the corresponding derivative 5 showed a 6-fold increase in potency (IC₅₀=0.79 μM).

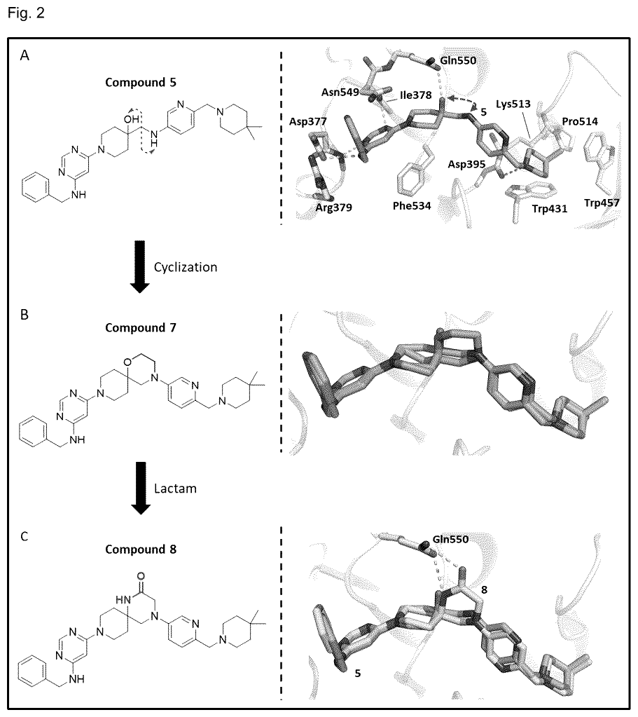

One striking feature of this inhibitor series is their linear shape coupled with sp³ acyclic atom linkers that makes them highly flexible. Rigidifying the structure is a viable way to freeze a ligand in its preferred conformation, which in turn can enhance the binding energy by reducing entropic penalties. Thus, the inventors envisioned two different strategies to achieve this goal: either making an amide connection between the piperidine and the pyridine ring, or according to compound 5's conformation, a spirocycle could be formed by connecting the tertiary alcohol with the aniline (FIG. 2A). The two methods brought opposite results, the amide derivative 6 lost the previous potency boost (IC_{50=3.6} μM, Table 1) while spirocycle 7 was promising both in terms of inhibition (IC_{50=0.28} μM, Table 2) and novelty. The inventors managed to soak both 5 and 7 with METTL3, and the X-ray analysis showed a strong structural overlap. The pyrimidine moiety is engaged in two hydrogen bonds with NH backbone from Asn549 and Ile378 while involved in Tr-stacking with Phe534 and π interactions with Asn549 side chain (FIG. 2A). The benzylamine group interacts with Asp377 side chain and also forms a cation-π interaction with Arg379. On the opposite site of the binding pocket, the gem dimethyl group fills a lipophilic pocket formed by Lys513, Pro514, Trp457 and Trp431 residues, whereas the charged piperidine forms a salt-bridge with Asp395. The sole difference between 5 and 7 is, for the latter, the missing hydrogen bond between the tertiary alcohol and GIn550 side chain due to the alcohol transformation into an ether (FIG. 2B). The inventors envisaged that replacing the ether by a lactam could restore this interaction and even make an additional hydrogen bond thanks to the C═O group of the ligand and the NH₂ amide of GIn550. The inventors obtained a strong potency boost for the corresponding derivative 8 (IC_{50=0.037} μM), and the inventor's hypothesis was confirmed by the two hydrogen bond interactions found in the crystal structure (FIG. 2C). Furthermore, both LE and LLE improved substantially (0.25 and 4.4 respectively, Table 2).

ADME properties, such as solubility, cell permeability, and metabolic stability are essential for chemical probes, so they were considered early on in the project. The inventor's newly synthesized inhibitors (5, 7-8) displayed mixed results; however, all of them displayed mediocre stability towards enzymatic degradation with half-lives lower than 12 minutes upon incubations with rat liver microsomes (Tables 1, 2). Therefore, the inventors focused on improving ADME properties while getting better biochemical potency. The initial approach was to substitute the pyridine nitrogen atom by a carbon atom, yielding 9 with moderate permeability (9·10⁻⁶ cm·s ⁻¹) and, surprisingly, slightly increased solubility (Table 2). However, metabolic stability remained unchanged, so the benzylamine was replaced with methylamine (10). Indeed, solubility and metabolic stability were significantly improved (108 μM and 107 min, respectively) as well as LE and LLE values (0.28 and 4.5, respectively), but at the expense of limited permeability (2·10⁻⁶ cm·s⁻¹) and a 3.4-fold potency reduction. From 10, two other possibilities of decreasing the size of the inventor's molecules were pursued: replacing spiropiperidine with spiroazetidine (11) and spirolactam with spirourea (12). Unfortunately, both displayed a substantial loss in potency (5 and 20 fold, respectively). Yet, the spiroazetidine moiety remains a potential alternative helping to reduce molecular weight and to improve physicochemical properties at a later stage. Next, the inventor's strategy was oriented towards permeability improvement. Lactam methylation in compound 13 resulted in a serious decrease in potency (19 fold), demonstrating the crucial role of the lactam hydrogen bond interactions.

After thorough spiro scaffold optimization, the inventors turned their attention to the pyrimidine motif. Addition of one more methyl on the aniline (14) was highly detrimental to binding compared to 10 (0.97 and 0.089 μM, respectively, Table 3), probably due to loss of the hydrogen bond to the side chain of Asp377, while methyl to isopropyl substitution (15) showed a less pronounced reduction as the hydrogen bond is preserved (0.33 μM). These two modifications illustrated the limited space available for branched spa carbons at this position. Surprisingly, substitution with a cyclopropyl group (16) was not only well tolerated (0.084 μM), but it also improved the three ADME properties (Table 3) and could become a promising alternative for lead optimization. S-Adenosyl methionine (SAM) is the natural ligand of METTL3 that contains an adenosine scaffold overlapping with the pyrimidine group of the inventor's inhibitors, thus the inventors thought to test a few bicyclic heteroaromatic modifications. The pyrrolopyrimydine 17 had a slight increase in potency in comparison to 10, but similarly low permeability and a larger efflux ratio in the Caco-2 assay (Table 3). The interaction geometry between the N₃ pyrimidine atom and Asn549 nitrogen backbone seemed not optimal, thus the inventors thought to remove this pyrimidine nitrogen atom in order to improve permeability and possibly to suppress a partial desolvation penalty. The latter proved to be false since pyrrolopyridine 18 exhibited a severe binding loss (74 fold). Incorporation of a chlorine atom between the two pyrimidine nitrogen atoms (19) was beneficial for potency (0.024 μM);

however, solubility and metabolic stability were critically impaired (45 μM and 32 min, respectively), which prompted the inventors to look for different modifications.

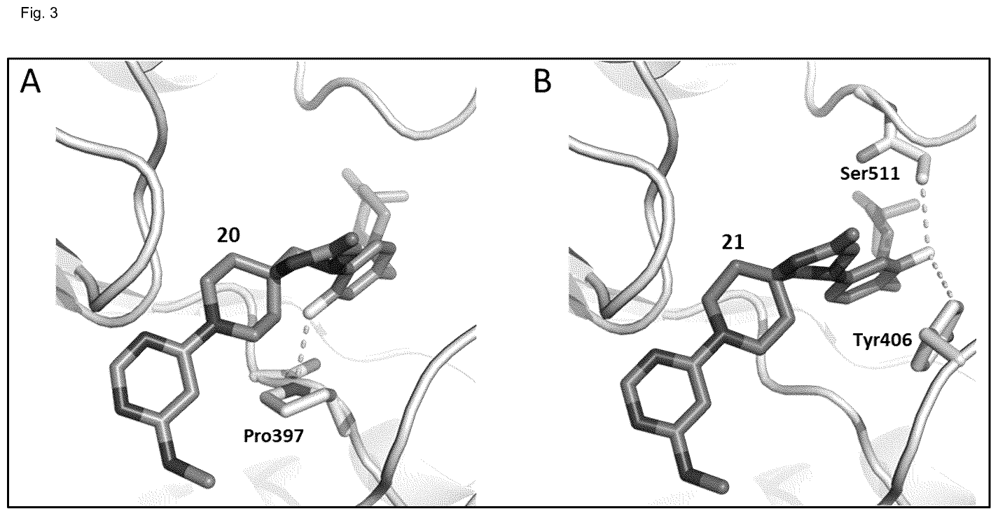

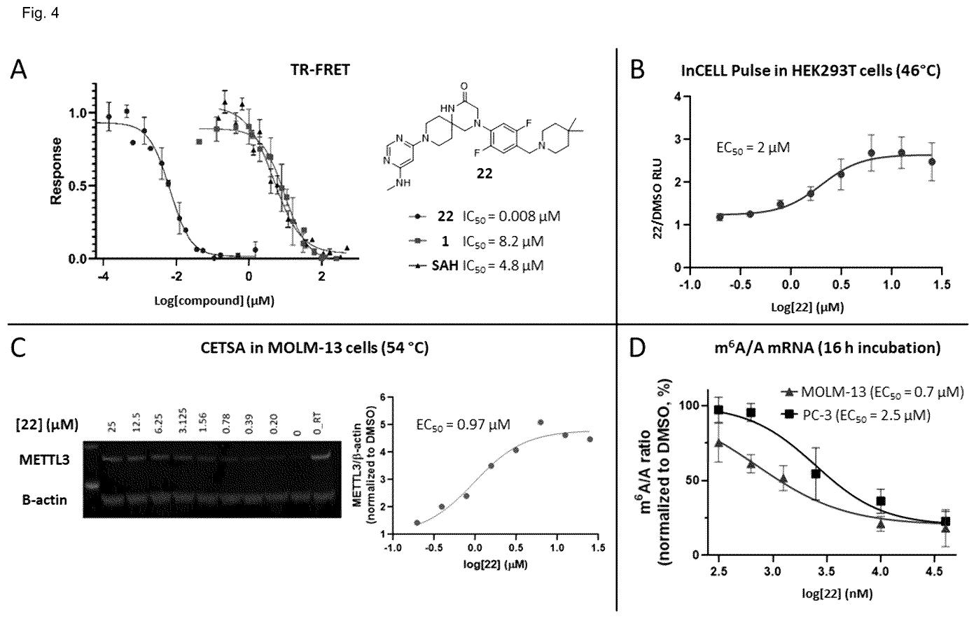

Because the spiro scaffold and the pyrimidine moiety were already optimized, the inventors considered the phenyl ring as the next target region. Several publications discuss the unique properties of fluorine atoms that can translate into unexpected and promising results in drug design. Indeed, fluorine atoms are able to make unusual interactions, and aromatic fluorine atoms tend to increase permeability. A fluorine scan was performed on the phenyl ring, affording two novel derivatives 20 and 21. Compared to the inhibitor 10, both compounds improved binding to a similar extent (0.038 and 0.032 μM, respectively); however, permeability was considerably increased only for 20 (Table 4). An X-ray structures in complex with METTL3 were solved for each molecule and revealed that the fluorine in 21 displays hydrophobic contacts (FIG. 3B), whereas the fluorine atom of 20 is also engaged in an unusual interaction with the nitrogen π system of Pro397 (FIG. 3A). Inhibitor 20 was preferable because of its strong improvement in permeability and small efflux ratio (9·10⁻⁶ cm·s⁻¹ and 2, respectively), but the combination of both fluorine atoms quickly emerged as the key solution to achieve excellent potency and to keep adequate ADME properties. Indeed, compound 22 exhibited single digit nanomolar IC₅₀ (0.008 μM) in the TR-FRET assay (Table 4 and FIG. 4A), high cell permeability (12·10⁻⁶ cm·s⁻¹), and favorable values of LE and LLE (0.3 and 5.3, respectively), as well as acceptable metabolic stability (t_{1/2}=24 min).

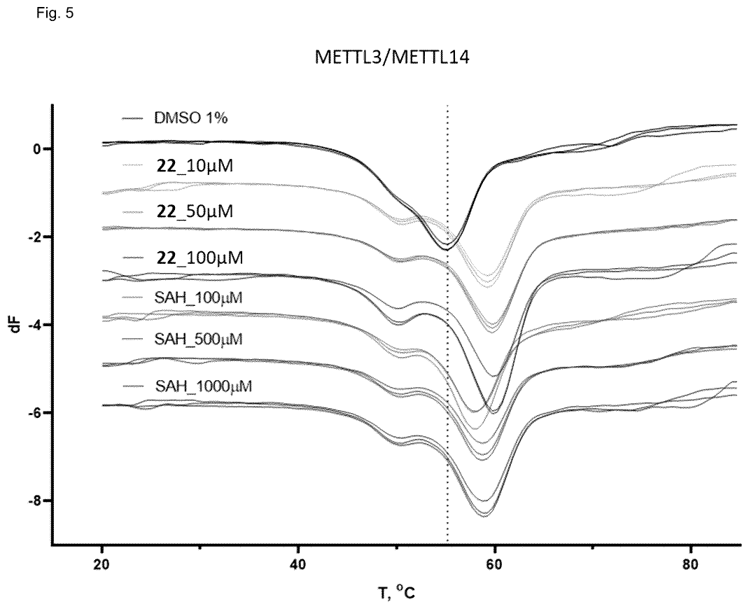

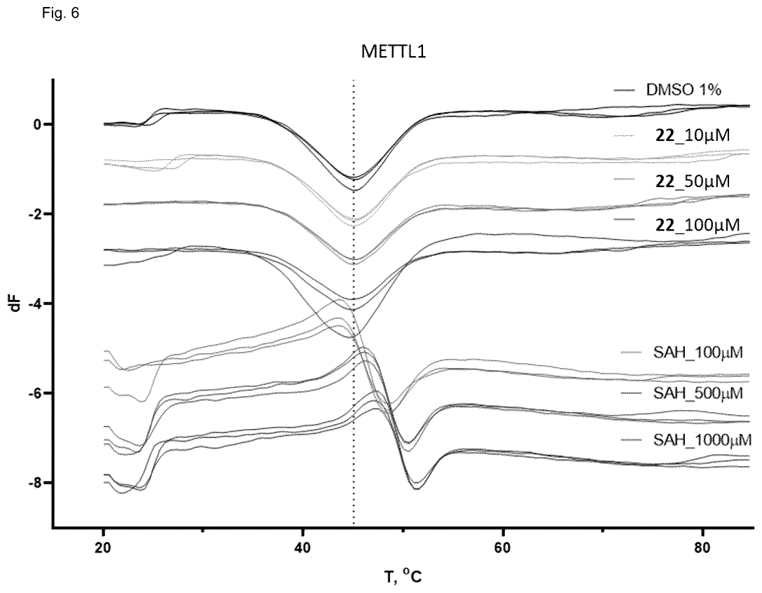

To investigate the selectivity of compound 22 towards other RNA methyltransferases, the inventors conducted protein thermal shift assay. The inventors expressed and purified METTL1 protein that is a writer of 7-methylguanosine mark on tRNA, mRNA, and miRNAs and serves as a representative closely related protein. The inventors employed as positive control S-adenosyl-L-homocysteine (SAH), a by-product of RNA methyltransferase catalytic activity and a natural binder, which showed ΔTₘ of 2.8° C. and 3.5° C. at 100 μM for METTL3/METTL14 and METTL1, respectively (FIGS. 5 and 6). Compound 22 at 100 μM was able to shift the melting temperature of METTL3/METTL14 by 4.7° C. compared to DMSO control (FIGS. 5 and 6). On the contrary, no shift was observed for METTL1 with compound 22 up to 100 μM indicating no binding.

The enhanced thermal stabilization of METTL3 by compound 22 allowed the inventors to study its cellular target engagement in two orthogonal assays based on protein thermal denaturation. The binding of 22 was evaluated in InCELL Pulse assay where enhanced ProLabel® (ePL) enzyme fragment fused to the N-terminus of the truncated METTL3 (residues 354-580) was expressed in HEK293T cells. After the incubation of these cells with inhibitor 22 for 1 h at 37° C., cells were heated at 46° C. for 3 min, and the non-aggregated METTL3-ePL protein was quantified using luminescence-based assay (FIG. 4B). Compound 22 stabilized the METTL3-ePL fusion protein with an EC₅₀ of 2 μM in HEK293T cells. Encouraged by these results, the inventors also conducted CETSA assay in MOLM-13 cells on an endogenously expressed full-length METTL3. Similarly to InCELL Pulse assay, CETSA in MOLM-13 cells demonstrated that 22 significantly stabilized METTL3 in a dose-dependent manner at 54° C. with EC₅₀ of 0.97 μM as determined by Western Blotting (FIG. 4C). Therefore, both experiments brought clear evidence of cell permeability and cellular target engagement. Finally, to highlight the biological potential of 22 as an inhibitor of METTL3 enzymatic activity, the inventors measured m⁶A/A ratio in polyadenylated RNA in two distinct cancer cell lines, MOLM-13 (AML) and PC-3 (prostate cancer) cells after 16 hours of compound treatment. The inventors found that 22 was able to reduce this ratio down to 10-20% of DMSO-treated control samples and with a certain degree of selectivity between the two cell lines (EC_{50=0.7} and 2.5 μM for MOLM-13 and PC-3 respectively, FIG. 4D).

The inventors successfully improved potency (by a factor of 1 000), efficiency parameters, and ADME properties of a series of METTL3 inhibitors by protein crystallography-guided medicinal chemistry. The key features were rigidification thanks to the design of spiro scaffolds and the use of fluorine atoms at specific positions. The most potent inhibitor (compound 22) shows an IC₅₀ of 8 nM in a TR-FRET assay. No binding to the off-target METTL1 was observed at concentrations of up to 100 μM. Cellular target engagement of compound 22 was demonstrated using two different assays. Furthermore, for the reduction of m⁶A/A in polyadenylated RNA, as quantified by UPLC-MS/MS analysis, EC₅₀ values of 0.7 μM and 2.5 μM were measured in MOLM-13 (leukemia) and PC-3 (prostate cancer) cell lines. Thus, compound 22 is a chemical probe to decipher the functional role of METTL3/METTL14 and its involvement in hematological malignancies and solid tumors.

TABLE 1

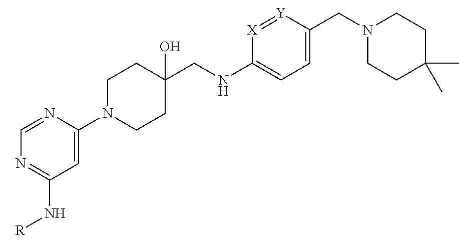

Early modifications of the original scaffold.

| | | | | | | | | Kinetic | Pₐₚₚ | |
|---|---|---|---|---|---|---|---|---|---|---|
| | | | | | | | | solu- | _{A-B} | |
| N^{o} | R | X | Y | IC₅₀¹ | MW² | LE³ | LLE⁴ | bility⁵ | (Efflux)⁶ | RLM⁷ |
| 1 | 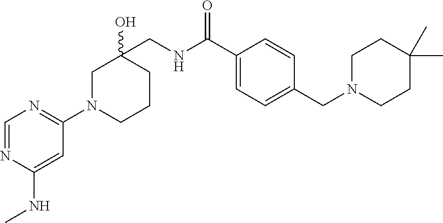 [SMILES: CNc1cc(N2CCCC(O)(CNC(=O)c3ccc(CN4CCC(C)(C)CC4)cc3)C2)ncn1] | | | 8.2 | 466.63 | 0.21 | 2.5 | — | — | — |
| 2 | Me | CH | CH | 5.0 | 438.62 | 0.23 | 2.5 | — | — | — |
| 3 | Me | N | CH | 4.6 | 439.61 | 0.23 | 3.2 | — | — | — |
| 4 | Me | CH | N | 5.8 | 439.61 | 0.22 | 3.4 | — | — | — |
| 5 | Bn | CH | N | 0.79 | 515.70 | 0.22 | 2.8 | 78 | 8 (5) | 5 |

| | | | | | | | | |
|---|---|---|---|---|---|---|---|---|
| 6 | 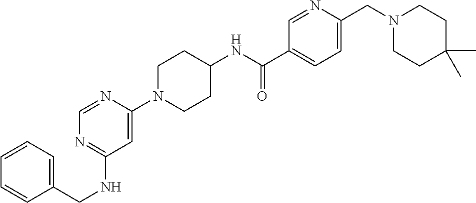 [SMILES: CC1(C)CCN(Cc2ccc(C(=O)NC3CCN(c4cc(NCc5ccccc5)ncn4)CC3)cn2)CC1] | 3.6 | 513.69 | 0.20 | 1.7 | — | — | — |

¹Time resolved-Förster resonance energy transfer (TR-FRET) assay (μM).
²g/mol.
³Ligand efficiency (kcal.mol⁻¹.heavy atom count⁻¹).
⁴Lipophilic ligand efficiency (pIC₅₀-logP);
⁵μM;
⁶10⁻⁶ cm.s⁻¹, (efflux ratio), Caco-2 experiment;
⁷Rat liver microsomes, t_{1/2} (min).

TABLE 2

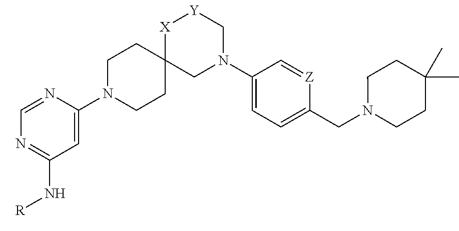

Derivatization from the spiro scaffold.

| | | | | | | | | | Kinetic | P_{app A-B} | |
|---|---|---|---|---|---|---|---|---|---|---|---|
| N^{o} | R | X | Y | Z | IC₅₀¹ | MW² | LE³ | LLE⁴ | solubility⁵ | (Efflux)⁶ | RLM⁷ |
| 7 | Bn | O | CH₂ | N | 0.28 | 541.74 | 0.22 | 2.8 | 61 | 15 (0.6) | 12 |
| 8 | Bn | NH | C═O | N | 0.037 | 554.74 | 0.25 | 4.4 | 44 | 2 (17) | 8 |
| 9 | Bn | NH | C═O | CH | 0.026 | 553.75 | 0.25 | 3.6 | 69 | 9 (3) | 9 |
| 10 | Me | NH | C═O | CH | 0.089 | 477.65 | 0.28 | 4.5 | 108 | 2 (9) | 107 |
| 11 | 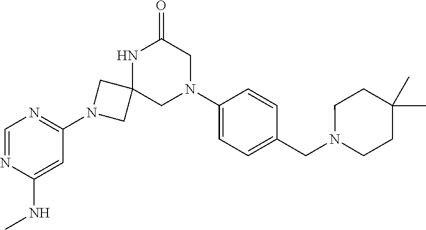 | | | | 0.44 | 449.60 | 0.26 | 4.5 | 86 | — | 70 |
| 12 | 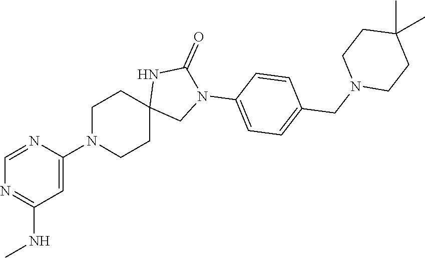 | | | | 1.8 | 463.63 | 0.23 | 2.6 | — | — | — |
| 13 | Me | NMe | C═O | CH | 1.5 | 491.68 | 0.22 | 3.0 | — | — | — |

TABLE 3

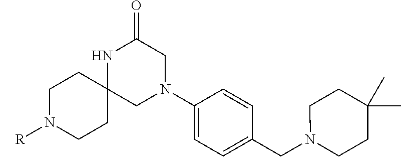

Optimization of the aminopyrimidine ring.

| Compound | | | | | | Kinetic | P_{app A-B} | |
|---|---|---|---|---|---|---|---|---|
| n^{o} | R | IC₅₀¹ | MW² | LE³ | LLE⁴ | solubility⁵ | (Efflux)⁶ | RLM⁷ |
| 14 | 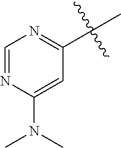 [SMILES: *c1cc(N(C)C)ncn1] | 0.97 | 491.68 | 0.23 | 3.2 | — | — | — |
| 15 | 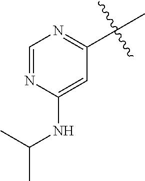 [SMILES: *c1cc(NC(C)C)ncn1] | 0.33 | 505.71 | 0.24 | 3.2 | — | — | — |
| 16 | 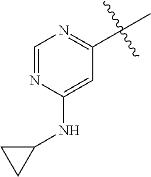 [SMILES: *c1cc(NC2CC2)ncn1] | 0.084 | 503.69 | 0.26 | 4.0 | 111 | 4 (7) | 118 |
| 17 | 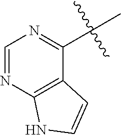 [SMILES: *c1ncnc2[nH]ccc12] | 0.061 | 487.65 | 0.27 | 4.3 | 73 | 2 (14) | 93 |
| 18 | 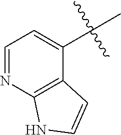 [SMILES: *c1ccnc2[nH]ccc12] | 4.5 | 486.66 | 0.20 | 2.3 | — | — | — |
| 19 | 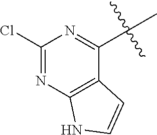 [SMILES: *c1nc(Cl)nc2[nH]ccc12] | 0.024 | 522.09 | 0.28 | 3.9 | 45 | — | 32 |

TABLE 4

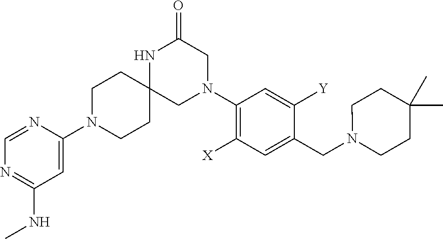

Fluorine scan on the phenyl ring.

| Compound | | | | | | | Kinetic | P_{app A-B} | |
|---|---|---|---|---|---|---|---|---|---|
| n^{o} | X | Y | IC₅₀¹ | MW² | LE³ | LLE⁴ | solubility⁵ | (Efflux)⁶ | RLM⁷ |
| 20 | F | H | 0.038 | 495.64 | 0.28 | 4.8 | 72 | 9 (2) | 63 |
| 21 | H | F | 0.032 | 495.64 | 0.29 | 4.8 | 92 | 3 (8) | 46 |
| 22 | F | F | 0.008 | 513.64 | 0.30 | 5.3 | 54 | 12 (2) | 24 |

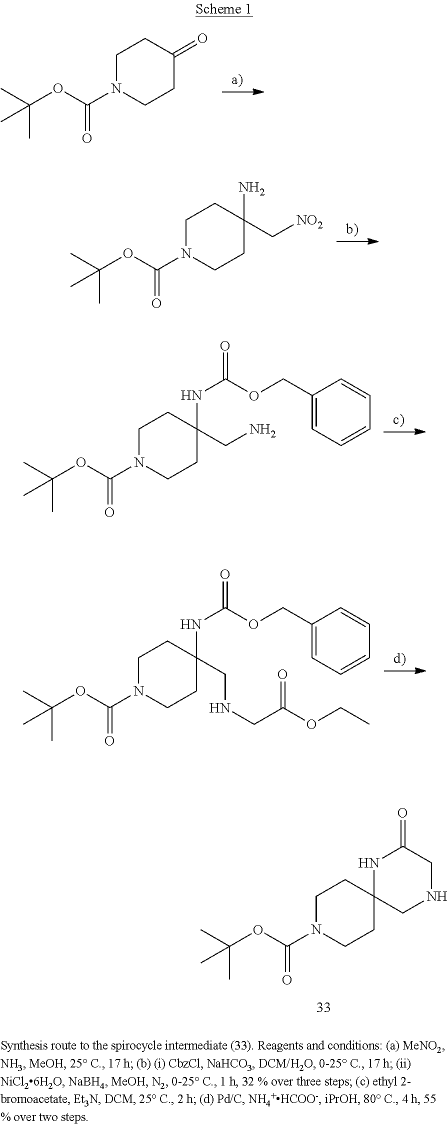

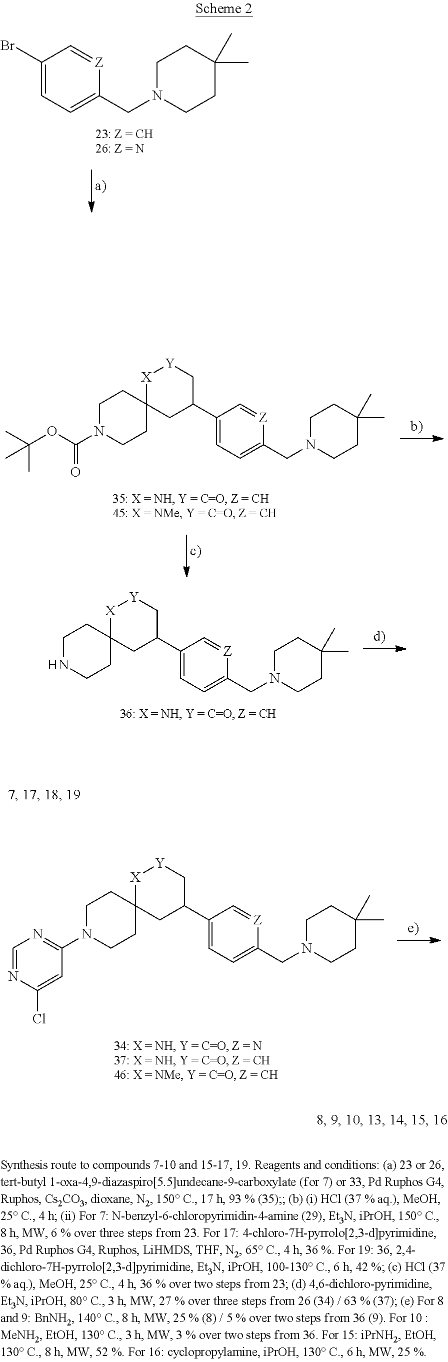

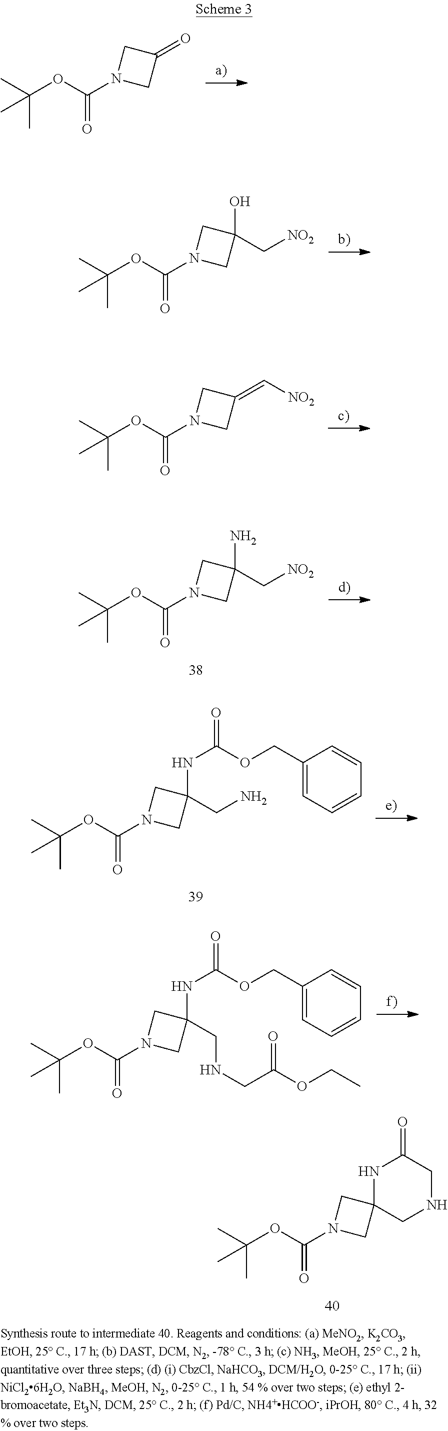

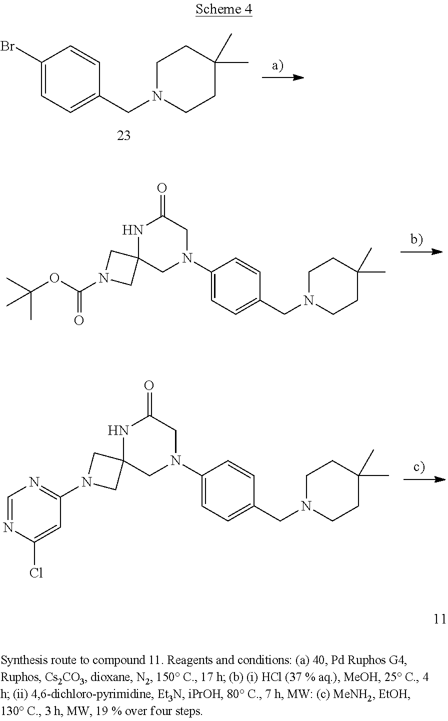

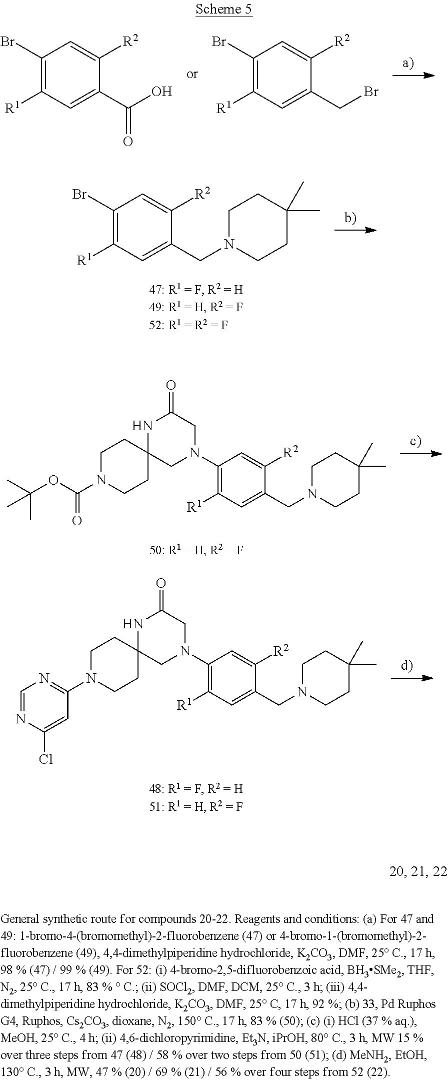

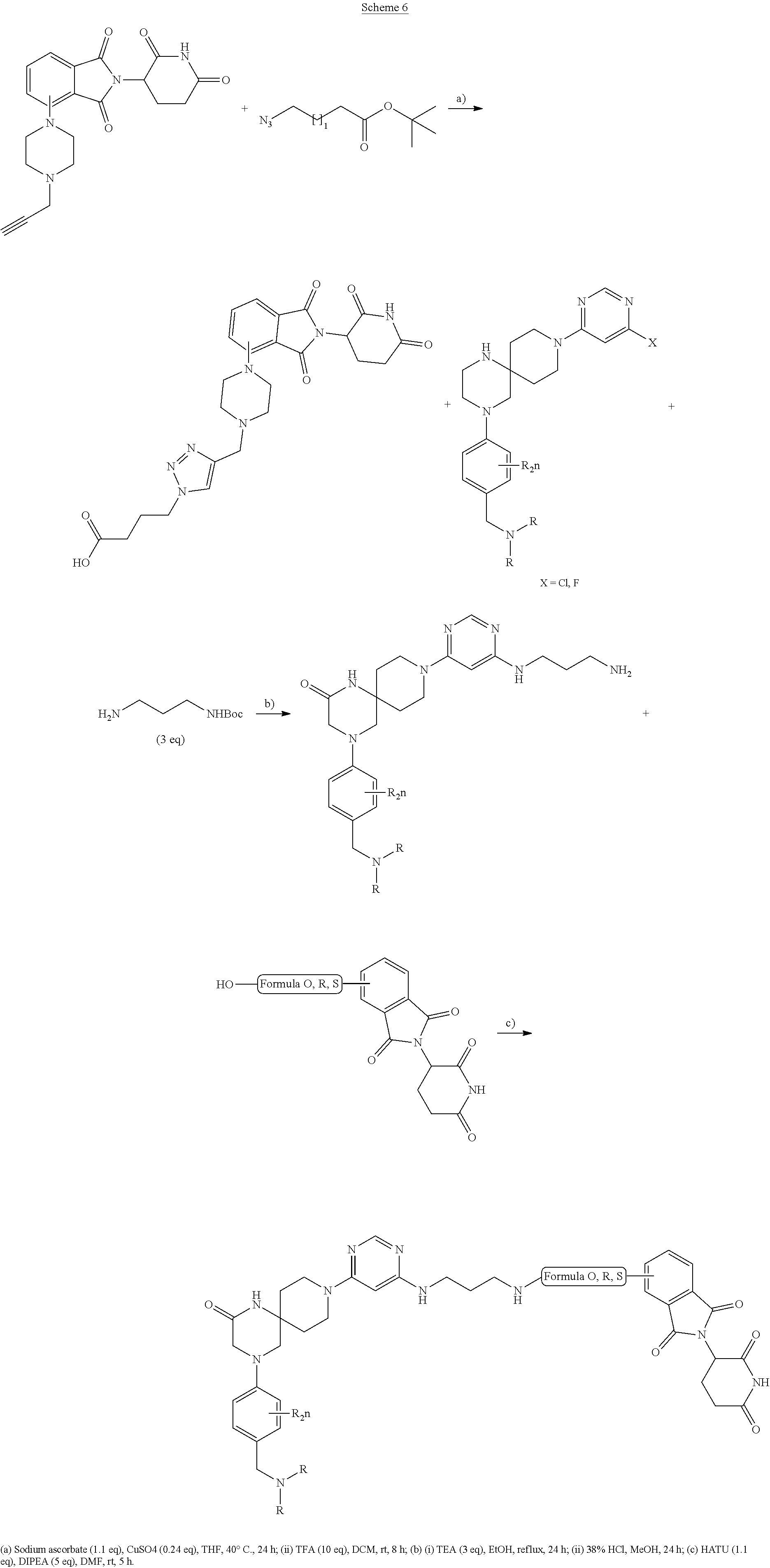

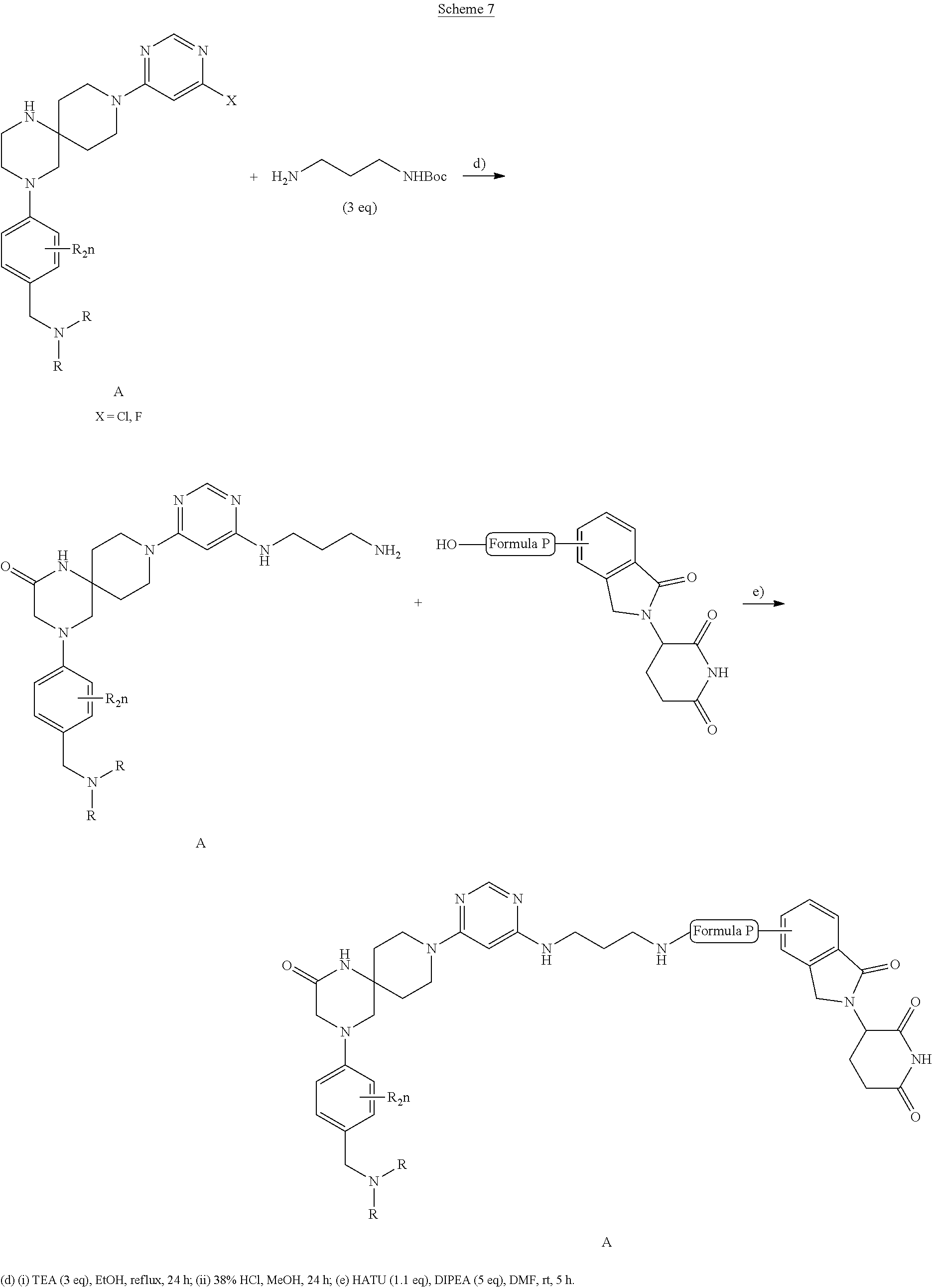

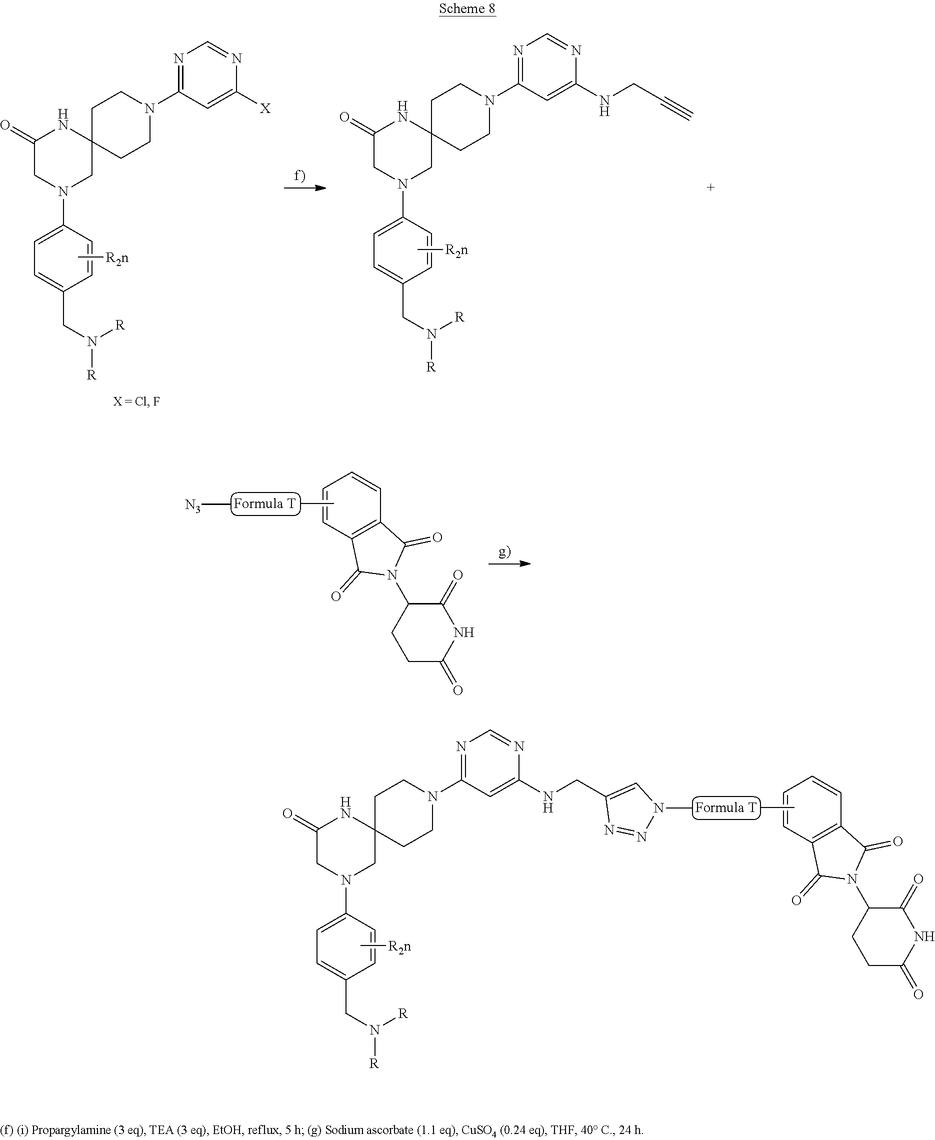

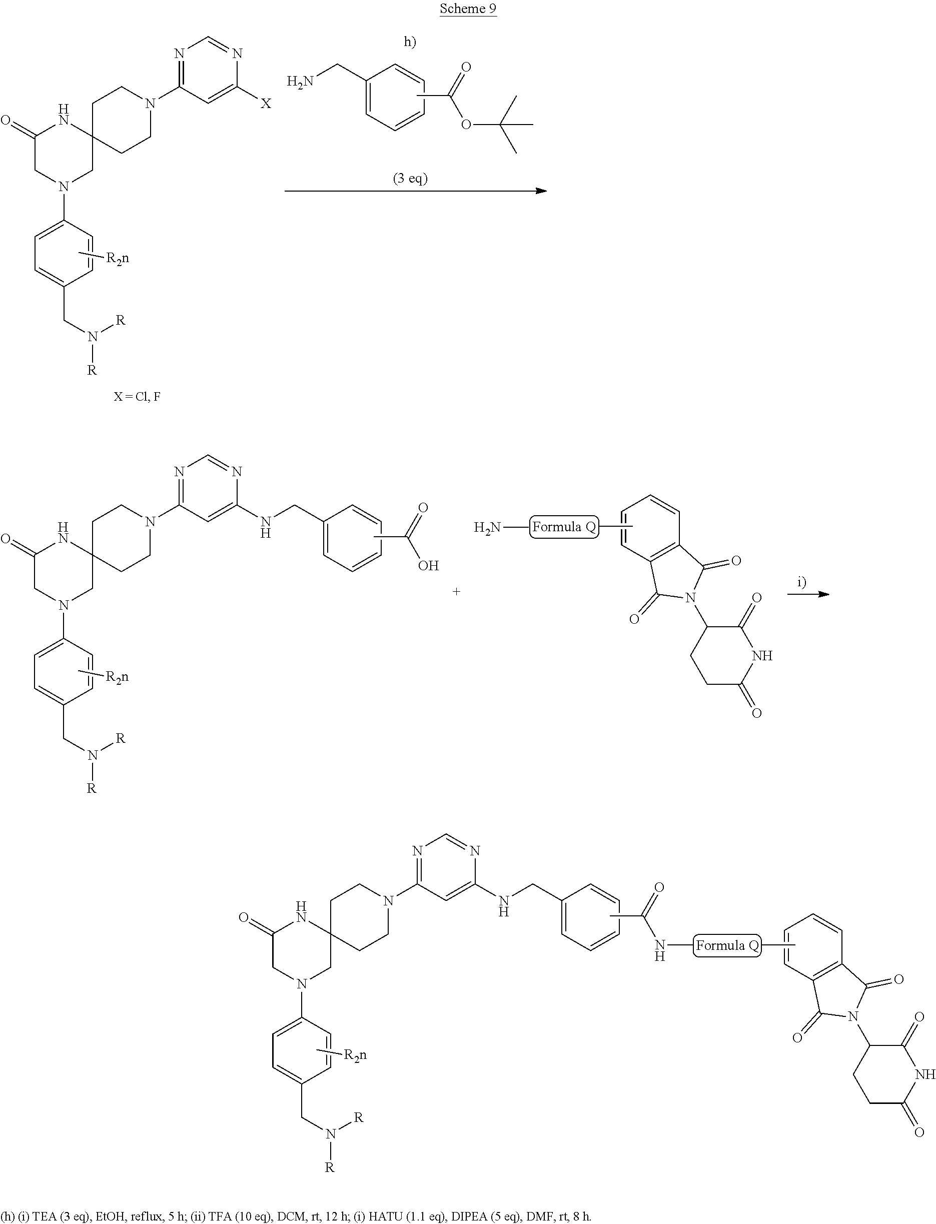

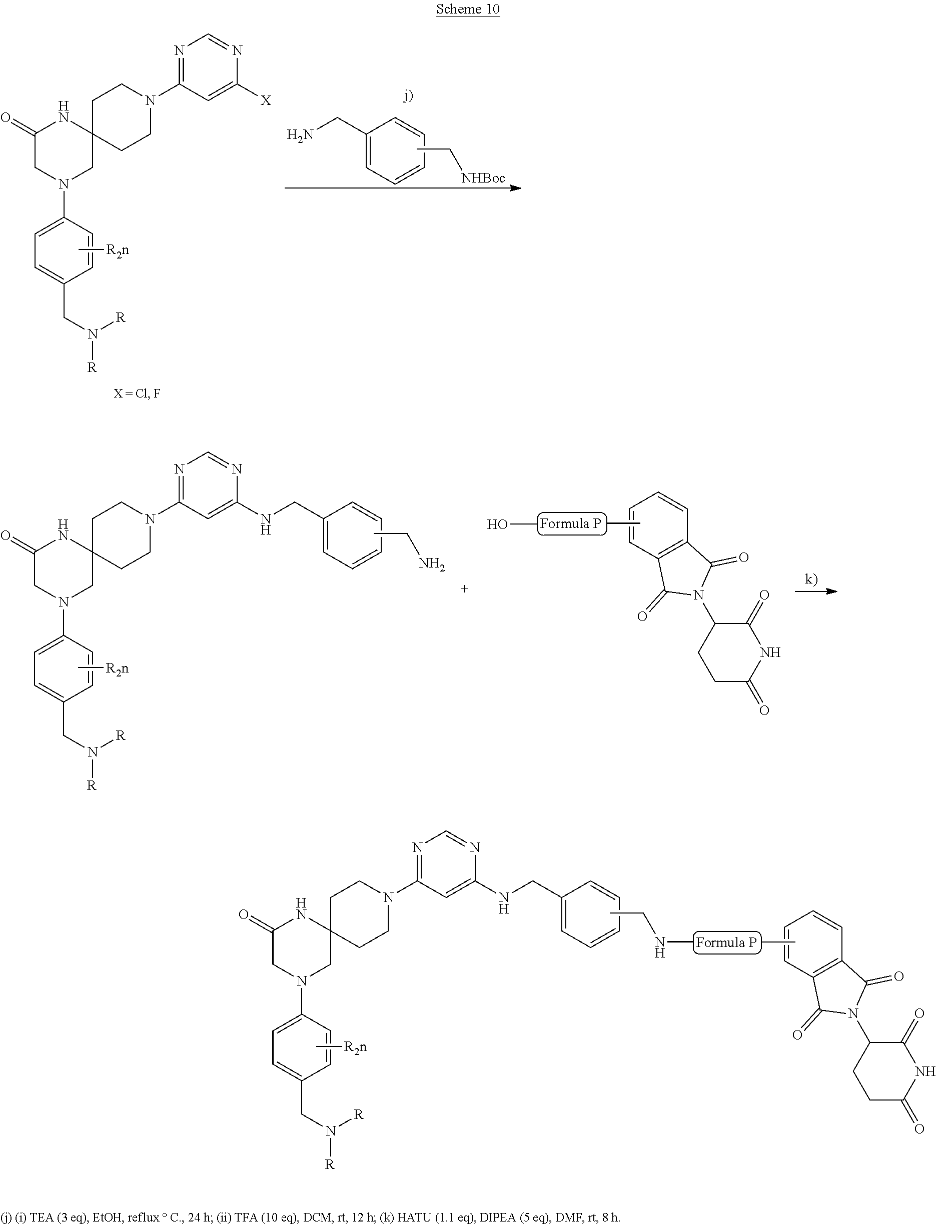

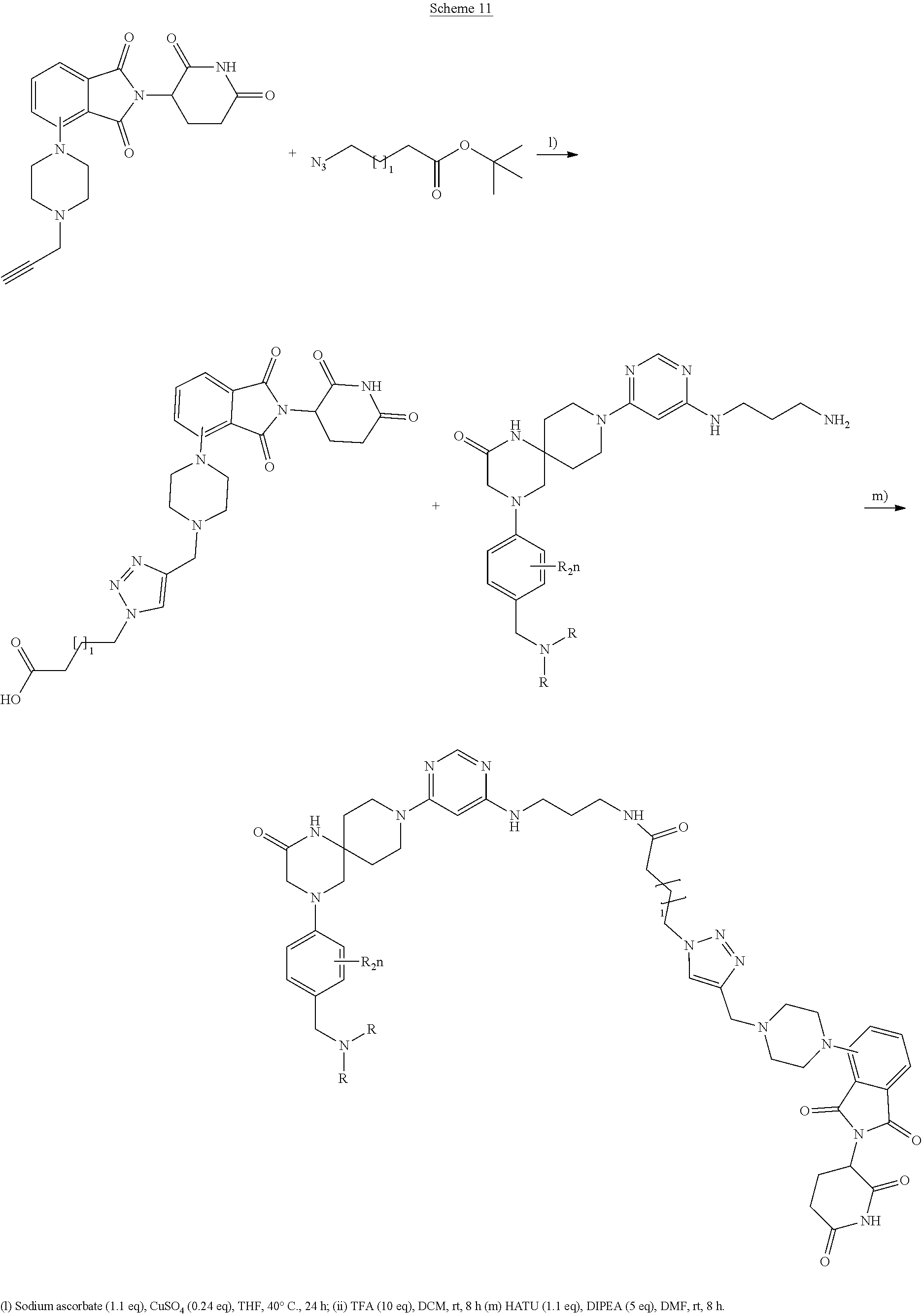

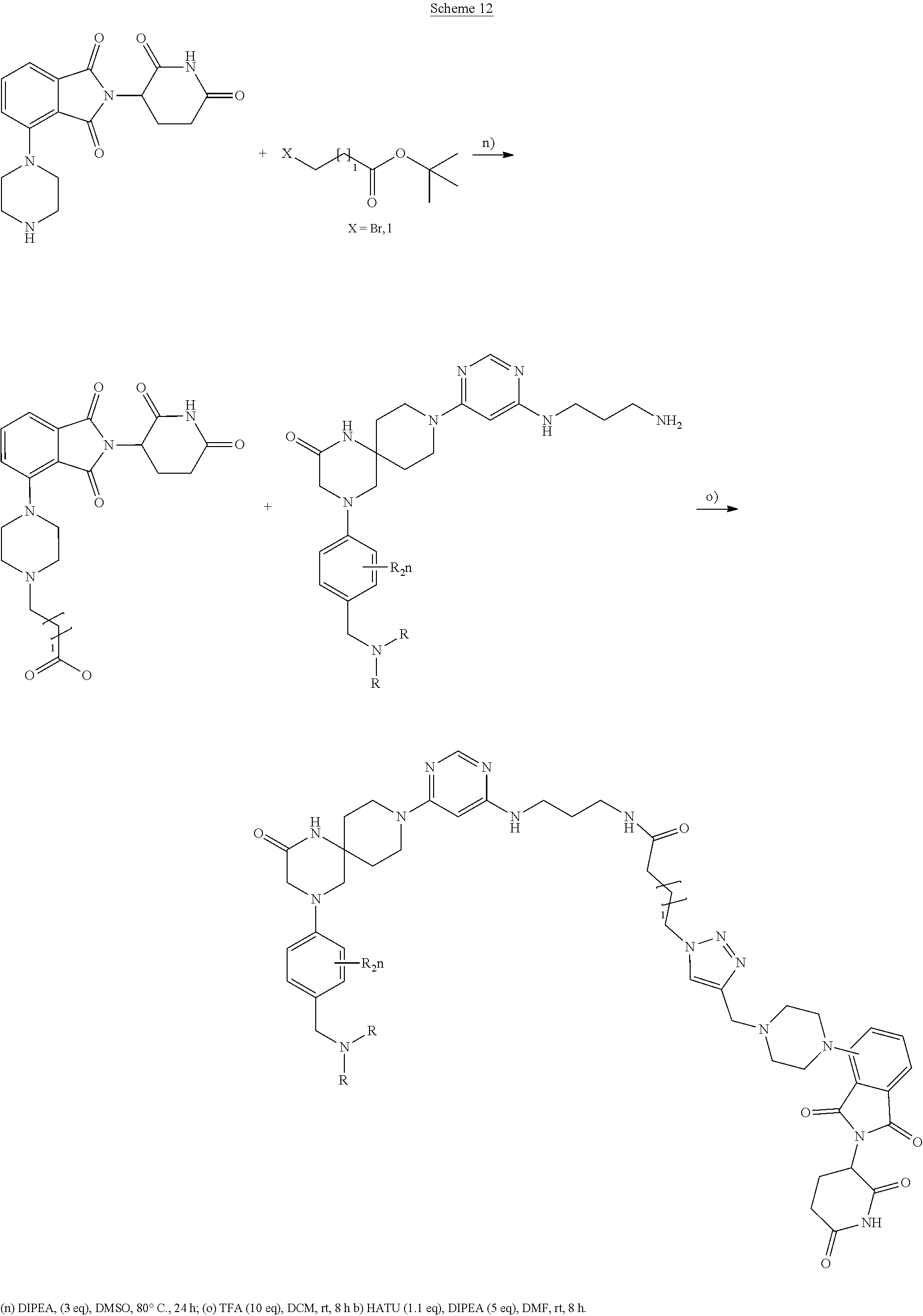

Synthesis schemes from literature incorporated by reference herein:

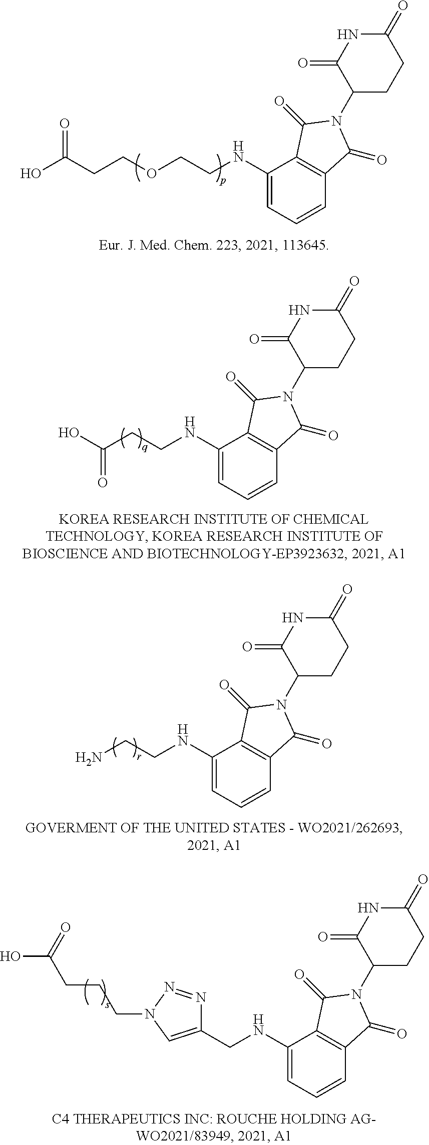

tert-Butyl 4-(aminomethyl)-4-(((benzyloxy)carbonyl)amino)piperidine-1-carboxylate (32):

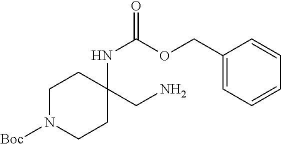 [SMILES: CC(C)(C)OC(=O)N1CCC(CN)(NC(=O)OCc2ccccc2)CC1]

To a stirred solution of MeNO₂ (1.3 equiv. 130 mmol, 7 mL) in NH₃ (53 mL, 7 N in MeOH), tert-butyl 4-oxopiperidine-1-carboxylate (20 g, 100 mmol) was added portionwise. The reaction mixture was stirred at 25° C. for 17 h and concentrated under reduced pressure. The crude residue was diluted with DCM and water. The two phases were separated and the aqueous layer was extracted two times with DCM. The combined organic layers were dried over MgSO₄, filtered and concentrated under reduced pressure to afford the desired product, which was engaged in the next step without further purification.

To a stirred solution of the corresponding amine (100 mmol) in dichloromethane (130 mL), a solution of K₂ CO₃ (2 equiv., 200 mmol, 27.6 g) in water (130 mL) was added. The reaction mixture was cooled to 0° C. and CBzCl (1.1 equiv., 110 mmol, 15.6 mL) was added dropwise. The reaction mixture was stirred at 25° C. for 17 h and the two phases were separated. The aqueous layer was extracted two times with DCM. The combined organic layers were washed once with brine, dried over MgSO₄, filtered and concentrated reduced pressure to afford the desired product, which was engaged in the next step without further purification.

To a stirred solution of the corresponding nitroalkane (100 mmol) in dry MeOH (450 mL), under a nitrogen atmosphere, at 0° C., NiCl₂·6 H₂O (1 equiv., 100 mmol, 27.3 g) was added, followed by NaBH₄ (5 equiv., 500 mmol, 18.9 g) portionwise to avoid strong H₂ evolution. Caution when adding NaBH₄, the reaction is highly exothermic and produce hydrogen gas. The reaction mixture was stirred at 25° C. for 1 h and quenched by adding saturated aqueous NaHCO₃ solution. The mixture was filtered through a pad of Celite, the filtrate was concentrated under reduced pressure and the obtained residue was diluted with water. The aqueous layer was extracted three times with DCM and the combined organic layers were washed once with brine, dried over MgSO₄, filtered and concentrated under reduced pressure. The crude residue was purified by flash column chromatography (DCM/MeOH/NH₄OH=100:0:0 to 100:3:0 to 100:3:1 to 100:5:1 to 100:10:1 to 100:15:1) to afford the desired product as a white solid (11.7 g, 32 yield over three steps). LRMS (ESI) m/z calcd for [C₁₉H₃₀N₃O₄]⁺: 364.2 found: 364.3

tert-Butyl 2-oxo-1,4,9-triazaspiro[5.5]undecane-9-carboxylate (33):

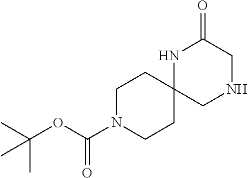 [SMILES: CC(C)(C)OC(=O)N1CCC2(CC1)CNCC(=O)N2]

To a stirred solution of tert-butyl 4-(aminomethyl)-4-(((benzyloxy)carbonyl)amino)piperidine-1-carboxylate (14.5 g, 40 mmol) in DCM (133 mL) at 0° C., Et₃N (0.8 equiv., 32 mmol, 4.4 mL) and ethyl 2-bromoacetate (0.7 equiv., 28 mmol, 3.1 mL) were added. The reaction mixture was stirred at 25° C. for 2 h and diluted with saturated aqueous NaHCO₃ solution. The aqueous layer was extracted three times with EtOAc and the combined organic layers were washed once with brine, dried over MgSO₄, filtered and concentrated under reduced pressure, to afford the impure desired product, which was engaged in the next step without further purification.

To a stirred solution of the corresponding Cbz protected amine (40 mmol) in iPrOH (400 mL), Pd/C (5 mol 2 mmol, 2.1 g, 10% wt) and ammonium formate (6 equiv., 240 mmol, 15 g) were added portionwise. The reaction mixture was stirred at 80° C. for 4 h, cooled to 25° C., filtered through a pad of celite and concentrated under reduced pressure. The obtained residue was dissolved in DCM, the organic layer was washed once with water, once with brine, dried over MgSO₄, filtered and concentrated under reduced pressure. The crude residue was purified by flash column chromatography (DCM/MeOH=100:5 to 100:8 to 100:10 to 100:15) to afford the desired product as a white solid (4.14 g, 55% yield over two steps). LRMS (ESI) m/z calcd for C₂₆H₄₇N₆O₆]⁺=[2 M+H]⁺: 539.4 found: 539.4

N-Benzyl-6-(4-(6-((4,4-dimethylpiperidin-1-yl)methyl)pyridin-3-yl)-1-oxa-4,9-diazaspiro[5.5]undecan-9-yl)pyrimidin-4-amine (7):

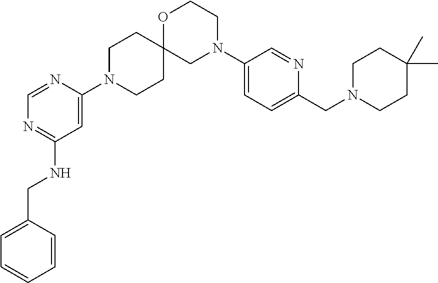 [SMILES: CC1(C)CCN(Cc2ccc(N3CCOC4(CCN(c5cc(NCc6ccccc6)ncn5)CC4)C3)cn2)CC1]

The corresponding Boc protected amine was obtained following the general procedure for Buchwald-Hartwig coupling (chromatography: EtOAc/heptane=7:3 to 9:1 to EtOAc/MeOH=100:0 to 100:1 to 100:5). The impure desired product was engaged in the next step without further purification.

The corresponding amine was obtained following the general procedure for Boc group deprotection. After evaporation, the crude residue was triturated in acetone and the resulting precipitate was filtered, washed with acetone and dried to afford the impure desired product, which was engaged in the next step without further purification. To a stirred solution of the corresponding amine (1 equiv.) in iPrOH (0.3 M), 29 (1.5 equiv.) and Et₃N (4 equiv.) were added. The reaction mixture was stirred at 150° C. for 8 h in the microwave and concentrated under reduced pressure. The reaction was diluted with water and the aqueous layer was extracted three times with DCM. The combined organic layers were washed five times with water, once with brine, dried over MgSO₄, filtered and concentrated under reduced pressure. The crude residue was purified by flash column chromatography (DCM/MeOH=100:5 to 100:8 to 100:10 to 100:13 to 100:20) to afford the desired product as a brown solid (6% yield over three steps). Mp: 61-62° C.; HRMS (ESI): m/z: calcd for [C₃₂ H₄₄ N₇O]⁺: 542.3607 found: 542.3602.

N-Benzyl-6-chloropyrimidin-4-amine (29):

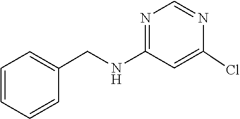 [SMILES: Clc1cc(NCc2ccccc2)ncn1]

To a stirred solution of 4,6-dichloro-pyrimidine (5 g, 33.6 mmol) in iPrOH (100 mL), benzylamine (1.2 equiv., 40.3 mmol, 4.4 mL) and Et₃N (1.2 equiv., 40.3 mmol, 5.59 mL) were added. The reaction mixture was stirred at 25° C. for 3 d and concentrated under reduced pressure. The crude residue was triturated in water, filtered and dried to afford the desired product as a beige solid (7.21 g, 98% yield). LRMS (ESI) m/z calcd for [C₁₁H₁₁ClN₃]⁺: 220.1 found: 220.1

9-(6-(Benzylamino)pyrimidin-4-yl)-4-(6-((4,4-dimethylpiperidin-1-yl)methyl)pyridin-3-yl)-1,4,9-triazaspiro[5.5]undecan-2-one (8):

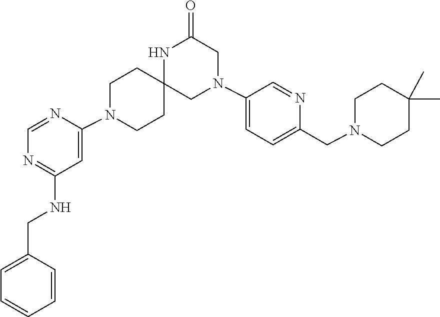 [SMILES: CC1(C)CCN(Cc2ccc(N3CC(=O)NC4(CCN(c5cc(NCc6ccccc6)ncn5)CC4)C3)cn2)CC1]

Compound 8 was obtained following the general procedure for S_{N}Ar with chloropyrimidine derivatives (chromatography: DCM/MeOH=100:5 to 100:10 to 100:15 to 100:20 to 100:30). The obtained impure product was triturated in diethyl ether, filtered, washed twice with ether, once with water and once with ether to afford the desired product as a white solid (14 mg, 25% yield). Mp: 208-209° C.; HRMS (ESI): m/z: calcd for [C₃₂ H₄₃N₈O]⁺: 555.3560 found: 555.3554.

9-(6-(Benzylamino)pyrimidin-4-yl)-4-(4-((4,4-dimethylpiperidin-1-yl)methyl)phenyl)-1,4,9-triazaspiro[5.5]undecan-2-one (9):

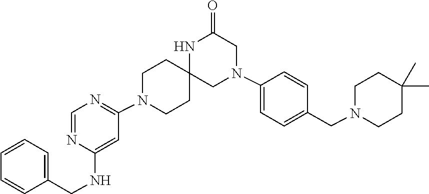 [SMILES: CC1(C)CCN(Cc2ccc(N3CC(=O)NC4(CCN(c5cc(NCc6ccccc6)ncn5)CC4)C3)cc2)CC1]

The corresponding chloropyrimidine was obtained following the general procedure for S_{N}Ar with 4,6-dichloropyrimidine. Instead of chromatography, after evaporation, the crude residue was triturated in water, filtered and washed once with water. The obtained sticky solid was dissolved in MeOH and concentrated under reduced pressure to afford the desired product, which was engaged in the next step without further purification.

Compound 9 was obtained following the general procedure for S_{N}Ar with chloropyrimidine derivatives (chromatography: DCM/MeOH=100:0 to 100:8 in 20 min, 100:8 for 10 min, 100:8 to 100:10 in 10 min). The obtained impure product was triturated in water, filtered, washed once with water to afford the desired product as a pale yellow solid (5% yield over two steps). Mp: 228-231° C.; HRMS (ESI): m/z: calcd for [C₃₃H₄₄N₇O]⁺: 554.3607 found: 554.3602.

4-(4-((4,4-dimethylpiperidin-1-yl)methyl)phenyl)-9-(6-(methylamino)pyrimidin-4-yl)-1,4,9-triazaspiro[5.5]undecan-2-one (10):

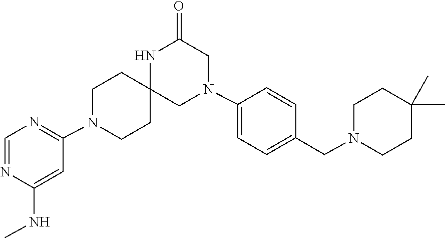

The corresponding chloropyrimidine batch was the same than the one used for compound 9.

Compound 10 was obtained following the general procedure for S_{N}Ar with chloropyrimidine derivatives (chromatography: DCM/MeOH=100:0 to 100:12 in 20 min, 100:12 for 10 min, 100:12 to 100:15 in 10 min, 100:15 for 10 min). White solid (3% yield over two steps). Mp: 235-236° C.; HRMS (ESI): m/z: calcd for [C₂₇H₄₀N₇O]⁺: 478.3294 found: 478.3289.

4-(4-((4,4-Dimethylpiperidin-1-yl)methyl)phenyl)-9-(7H-pyrrolo[2,3-d]pyrimidin-4-yl)-1,4,9-triazaspiro[5.5]undecan-2-one (17):

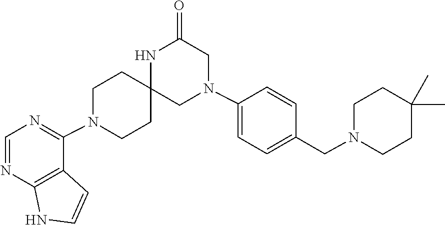 [SMILES: CC1(C)CCN(Cc2ccc(N3CC(=O)NC4(CCN(c5ncnc6[nH]ccc56)CC4)C3)cc2)CC1]

To a stirred solution of 36 (100 mg, 0.21 mmol) in dry THF (700 μL), under a nitrogen atmosphere, 4-chloro-7H-pyrrolo[2,3-d]pyrimidine (1 equiv., 0.21 mmol, 32 mg) was added. Nitrogen gas was bubbled through the reaction for two minutes and Ruphos Pd G4 (5 mol %, 0.011 mmol, 8.9 mg), Ruphos (5 mol %, 0.011 mmol, 5.1 mg) and LiHMDS (6.6 equiv., 1.39 mmol, 1.39 mL, 1 M THF) were added. The reaction mixture was stirred at 65° C. for 4 h, cooled down to 25° C. and concentrated under reduced pressure. The crude residue was purified by flash column chromatography (DCM/MeOH=100:3 to 100:5 to 100:8 to 100:10 to 100:15) to afford the desired product as a yellow solid (37 mg, 36% yield). Mp: 250-252° C.; HRMS (ESI): m/z: calcd for [C₂₈ H₃₈ NO]⁺: 488.3138. found: 488.3132.

9-(2-Chloro-7H-pyrrolo[2,3-d]pyrimidin-4-yl)-4-(4-((4,4-dimethylpiperidin-1-yl)methyl)phenyl)-1,4,9-triazaspiro[5.5]undecan-2-one (19):

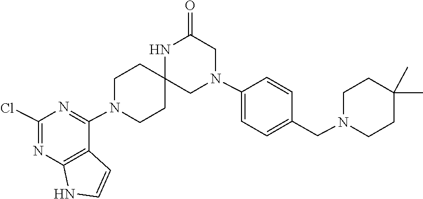 [SMILES: CC1(C)CCN(Cc2ccc(N3CC(=O)NC4(CCN(c5nc(Cl)nc6[nH]ccc56)CC4)C3)cc2)CC1]

To a stirred solution of 36 (100 mg, 0.21 mmol) in iPrOH (1 mL), 2,4-dichloro-7H-pyrrolo[2,3-d]pyrimidine (1.2 equiv., 0.25 mmol, 47 mg) and Et₃N (4 equiv., 0.84 mmol, 116 μL) were added. The reaction mixture was stirred at 100° C. for 3 h and an additional 3 h at 130° C., both in the microwave. The reaction mixture was concentrated under reduced pressure and the crude residue was triturated in water. The resulting precipitate was filtered, washed with water and dried to afford the desired impure product, which was further purified by flash column chromatography (DCM/MeOH=100:0 to 100:10 in 15 min, 100:10 for 10 min) to afford the desired product as a beige solid (46 mg, 42 yield). Mp: 199-201° C.; HRMS (ESI): m/z: calcd for [C₂₈ H₃₇ ClN₇O]⁺: 522.2748 found: 522.2731.

9-(6-(Cyclopropylamino)pyrimidin-4-yl)-4-(4-((4,4-dimethylpiperidin-1-yl)methyl)phenyl)-1,4,9-triazaspiro[5.5]undecan-2-one (16):

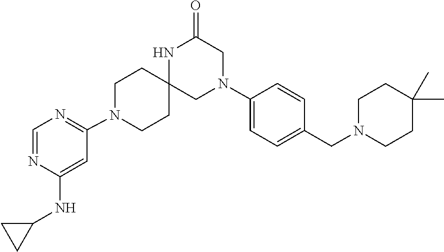 [SMILES: CC1(C)CCN(Cc2ccc(N3CC(=O)NC4(CCN(c5cc(NC6CC6)ncn5)CC4)C3)cc2)CC1]

Compound 16 was obtained following the general procedure for S_{N}Ar with chloropyrimidine derivatives (chromatography: DCM/MeOH=100:0 to 100:10 in 15 min, 100:10 for 10 min, 100:10 to 100:15 in 15 min). Brown solid (25% yield). Mp: 172-174° C.; HRMS (ESI): m/z: calcd for [C₂₉ H₄₂N₇O]⁺: 504.3451 found: 504.3445.

4-(4-((4,4-Dimethylpiperidin-1-yl)methyl)phenyl)-9-(6-(isopropylamino)pyrimidin-4-yl)-1,4,9-triazaspiro[5.5]undecan-2-one (15):

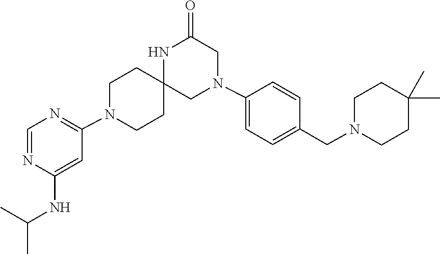 [SMILES: CC(C)Nc1cc(N2CCC3(CC2)CN(c2ccc(CN4CCC(C)(C)CC4)cc2)CC(=O)N3)ncn1]

Compound 15 was obtained following the general procedure for S_{N}Ar with chloropyrimidine derivatives (chromatography: DCM/MeOH=100:0 to 100:8 in 15 min, 100:8 for 10 min, 100:8 to 100:10 in 10 min). Beige solid (52% yield). Mp: 175-176° C.; HRMS (ESI): m/z: calcd for [C₂₉ H₄₄N₇O]⁺: 506.3607 found: 506.3602.

1-(4-Bromobenzyl)-4,4-dimethylpiperidine (23):

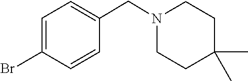 [SMILES: CC1(C)CCN(Cc2ccc(Br)cc2)CC1]

Intermediate 23 was obtained following the general procedure for dimethylpiperidine alkylation (chromatography: EtOAc/heptane=0:10 to 3:7). Yellow oil, 99% yield. LRMS (ESI) m/z calcd for [C₁₄H₂₁BrN]⁺: 282.1 found: 282.1.

5-Chloro-2-((4,4-dimethylpiperidin-1-yl)methyl)pyridine (26):

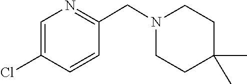 [SMILES: CC1(C)CCN(Cc2ccc(Cl)cn2)CC1]

To a stirred solution of (5-chloropyridin-2-yl)methanol (2.43 g, 17 mmol) in DCM (40 mL), SOCl₂ (1.5 equiv., 25.5 mmol, 1.85 mL) and DMF (1 drop) were added. The reaction mixture was stirred at 25° C. for 2 h and concentrated under reduced pressure to afford the chloroalkane, which was engaged in the next step without further purification.

Intermediate 26 was obtained following the general procedure for dimethylpiperidine alkylation but the reaction mixture was stirred at 70° C. for 3 h (column chromatography: EtOAc/heptane=1:9 to 3:7). Yellow solid, 86% yield over two steps. LRMS (ESI) m/z calcd for [C₁₃H₂₀ ClN₂]⁺: 239.1 found: 239.2

9-(6-Chloropyrimidin-4-yl)-4-(6-((4,4-dimethylpiperidin-1-yl)methyl)pyridin-3-yl)-1,4,9-triazaspiro[5.5]undecan-2-one (34):

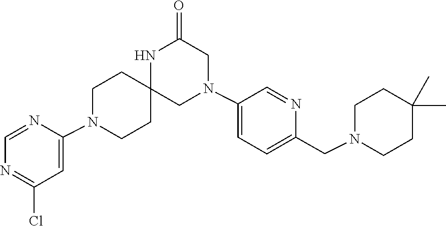 [SMILES: CC1(C)CCN(Cc2ccc(N3CC(=O)NC4(CCN(c5cc(Cl)ncn5)CC4)C3)cn2)CC1]

The corresponding Boc protected amine was obtained following the general procedure for Buchwald-Hartwig coupling (chromatography: DCM/MeOH=100:2 to 100:5 to 100:8 to 100:12 to 100:15 to 100:20). The impure desired product was engaged in the next step without further purification.

The corresponding amine was obtained following the general procedure for Boc group deprotection. After evaporation, the crude residue was triturated in acetone and the resulting precipitate was filtered, washed with acetone and dried to afford the impure desired product, which was engaged in the next step without further purification.

Intermediate 34 was obtained following the general procedure for S_{N}Ar with 4,6-dichloropyrimidine (column chromatography: DCM/MeOH=100:2 to 100:5 to 100.8 to 100:10 to 100:15 to 100:20) to afford the desired product as a yellow solid (123 mg, 27 yield over three steps). LRMS (ESI) m/z calcd for [C₂₅ H₃₅ ClN₇O]⁺: 484.3 found: 484.3.

tert-Butyl 4-(4-((4,4-dimethylpiperidin-1-yl)methyl)phenyl)-2-oxo-1,4,9-triazaspiro[5.5]undecane-9-carboxylate (35):

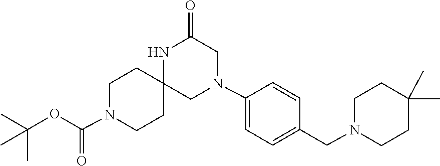 [SMILES: CC1(C)CCN(Cc2ccc(N3CC(=O)NC4(CCN(C(=O)OC(C)(C)C)CC4)C3)cc2)CC1]

Intermediate 35 was obtained following the general procedure for Buchwald-Hartwig coupling (chromatography: DCM/MeOH=100:5 to 100:8 to 100:12 to 100:20). Beige solid, 93% yield.

4-(4-((4,4-Dimethylpiperidin-1-yl)methyl)phenyl)-1,4,9-triazaspiro[5.5]undecan-2-one hydrochloride (36):

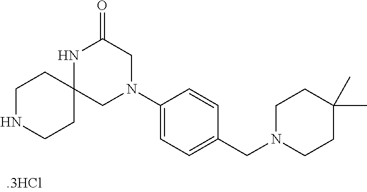 [SMILES: CC1(C)CCN(Cc2ccc(N3CC(=O)NC4(CCNCC4)C3)cc2)CC1]

Intermediate 36 was obtained following the general procedure for Boc group deprotection. After evaporation, the crude residue was triturated in acetone and the resulting precipitate was filtered, washed with acetone and dried to afford the desired product as a beige solid (50% yield over two steps from intermediate 32). LRMS (ESI) m/z calcd for [C₂₂ H₃₅ N₄O]⁺: 371.3 found: 371.3.

9-(6-Chloropyrimidin-4-yl)-4-(4-((4,4-dimethylpiperidin-1-yl)methyl)phenyl)-1,4,9-triazaspiro[5.5]undecan-2-one (37):

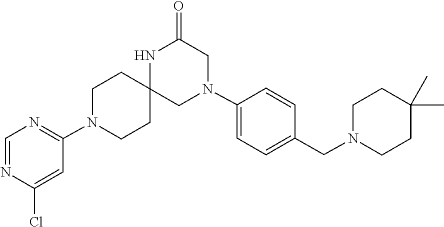 [SMILES: CC1(C)CCN(Cc2ccc(N3CC(=O)NC4(CCN(c5cc(Cl)ncn5)CC4)C3)cc2)CC1]

Intermediate 37 was obtained following the general procedure for S_{N}Ar with 4,6-dichloropyrimidine. Instead of chromatography, after evaporation of the crude mixture, the residue was triturated in water. The obtained precipitate was filtered, washed with water and dried to afford the desired product as a brown solid (63% yield). LRMS (ESI) m/z calcd for [C₂₆ H₃₆ ClN₆O]⁺: 483.3 found: 483.3.

4-(4-((4,4-Dimethylpiperidin-1-yl)methyl)-2-fluorophenyl)-9-(6-(methylamino)pyrimidin-4-yl)-1,4,9-triazaspiro[5.5]undecan-2-one (20):

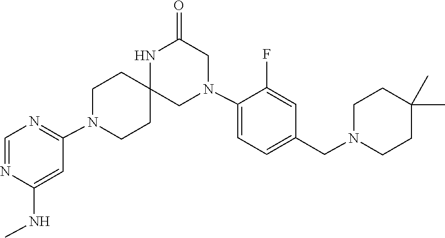

Compound 20 was obtained following the general procedure for S_{N}Ar with chloropyrimidine derivatives (chromatography: DCM/MeOH=100:0 to 100:8 in 20 min, 100:8 for 15 min). White solid (47% yield). Mp: 202-204° C.; HRMS (ESI): m/z: calcd for [C₂₇H₃₉FN₇O]⁺: 496.3200 found: 496.3195.

4-(4-((4,4-Dimethylpiperidin-1-yl)methyl)-3-fluorophenyl)-9-(6-(methylamino)pyrimidin-4-yl)-1,4,9-triazaspiro[5.5]undecan-2-one (21):

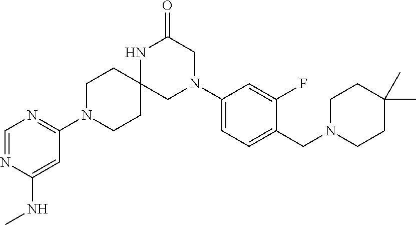

Compound 21 was obtained following the general procedure for S_{N}Ar with chloropyrimidine derivatives (chromatography: DCM/MeOH=100:0 to 100:8 in 20 min, 100:8 for 5 min, 100:8 to 100:12 in 10 min, 100:12 for 5 min). Yellow solid, 69% yield. Mp: 210-211° C.; HRMS (ESI): m/z: calcd for [C₂₇H₃₉FN₇O]⁺: 496.3200 found: 496.3195.

4-(4-((4,4-Dimethylpiperidin-1-yl)methyl)-2,5-difluorophenyl)-9-(6-(methylamino)pyrimidin-4-yl)-1,4,9-triazaspiro[5.5]undecan-2-one (22, UZH2):

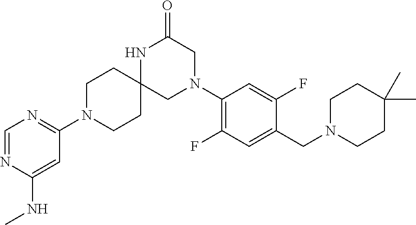

The corresponding Boc protected amine was obtained following the general procedure for Buchwald-Hartwig coupling (chromatography: DCM/MeOH=100:3 to 100:5 to 100:8). The impure desired product was engaged in the next step without further purification.

The corresponding amine was obtained following the general procedure for Boc group deprotection. The impure desired product was engaged in the next step without further purification.

The corresponding chloropyrimidine was obtained following the general procedure for S_{N}Ar with 4,6-dichloropyrimidine. The impure desired product was engaged in the next step without further purification.

UZH2 was obtained following the general procedure for S_{N}Ar with chloropyrimidine derivatives (chromatography: DCM/MeOH=100:0 to 100:10 in 15 min, to 100:10 for 10 min, 100:10 to 100:12 in 10 min. White solid, 56% yield over four steps. Mp: 214-216; HRMS (ESI): m/z: calcd for [C₂₇H₃₈F₂N₇O]⁺: 514.3106 found: 514.3100.

1-(4-Bromo-3-fluorobenzyl)-4,4-dimethylpiperidine (47):

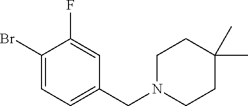 [SMILES: CC1(C)CCN(Cc2ccc(Br)c(F)c2)CC1]

Intermediate 47 was obtained following the general procedure for dimethylpiperidine alkylation (column chromatography: EtOAc/heptane=1:9). Colorless oil, 98% yield. LRMS (ESI) m/z calcd for [C₁₄ H₂₀ BrFN]⁺: 300.1 found: 300.1.

9-(6-Chloropyrimidin-4-yl)-4-(4-((4,4-dimethylpiperidin-1-yl)methyl)-2-fluorophenyl)-1.4.9-triazaspiro[5.5]undecan-2-one (48):

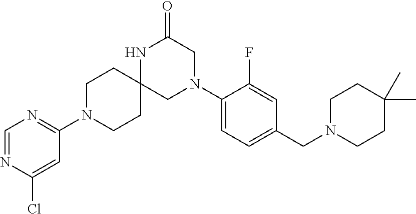 [SMILES: CC1(C)CCN(Cc2ccc(N3CC(=O)NC4(CCN(c5cc(Cl)ncn5)CC4)C3)c(F)c2)CC1]

The corresponding Boc protected amine was obtained following the general procedure for Buchwald-Hartwig coupling (chromatography: DCM/MeOH=100:3 to 100:5 to 100:8). The impure desired product was engaged in the next step without further purification.

The corresponding amine was obtained following the general procedure for Boc group deprotection. The impure desired product was engaged in the next step without further purification.

Intermediate 48 was obtained following the general procedure for S_{N}Ar with 4,6-dichloropyrimidine (column chromatography: DCM/MeOH=100:3 to 100:5 to 100.8 to 100:10) to afford the desired product as a white solid (15% yield over three steps). LRMS (ESI) m/z calcd for [C₂₆ H₃₅ ClFN₆O]⁺: 501.3 found: 501.3.

1-(4-Bromo-2-fluorobenzyl)-4,4-dimethylpiperidine (49):

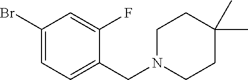 [SMILES: CC1(C)CCN(Cc2ccc(Br)cc2F)CC1]

Intermediate 49 was obtained following the general procedure for dimethylpiperidine alkylation. Colorless liquid, 99% yield. LRMS (ESI) m/z calcd for [C₁₄H₂₀BrFN]⁺: 300.1 found: 300.1.

tert-Butyl 4-(4-((4,4-dimethylpiperidin-1-yl)methyl)-3-fluorophenyl)-2-oxo-1,4,9-triazaspiro[5.5]undecane-9-carboxylate (50):

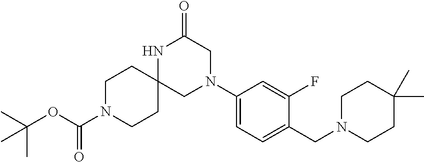 [SMILES: CC1(C)CCN(Cc2ccc(N3CC(=O)NC4(CCN(C(=O)OC(C)(C)C)CC4)C3)cc2F)CC1]

Intermediate 50 was obtained following the general procedure for Buchwald-Hartwig coupling (chromatography: DCM/MeOH=100:5 to 100:8 to 100:12 to 100:16). Brown solid (83% yield). LRMS (ESI) m/z calcd for [C₂₇H₄₂FN₄O₃]⁺: 489.3 found: 489.4.

9-(6-Chloropyrimidin-4-yl)-4-(4-((4,4-dimethylpiperidin-1-yl)methyl)-3-fluorophenyl)-1,4,9-triazaspiro[5.5]undecan-2-one (51):

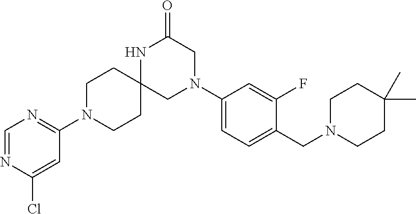 [SMILES: CC1(C)CCN(Cc2ccc(N3CC(=O)NC4(CCN(c5cc(Cl)ncn5)CC4)C3)cc2F)CC1]

The corresponding amine was obtained following the general procedure for Boc group deprotection. The impure desired product was engaged in the next step without further purification.

Intermediate 51 was obtained following the general procedure for S_{N}Ar with 4,6-dichloropyrimidine. Instead of chromatography, after evaporation of the crude mixture, the residue was triturated in water. The obtained precipitate was filtered, washed with water and dried to afford the desired product as a brown solid (58 yield over two steps). LRMS (ESI) m/z calcd for [C₂₆ H₃₅ ClFN₆O]⁺: 501.3 found: 501.3.

1-(4-Bromo-2,5-difluorobenzyl)-4,4-dimethylpiperidine (52):

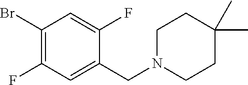 [SMILES: CC1(C)CCN(Cc2cc(F)c(Br)cc2F)CC1]

To a stirred solution of 4-bromo-2,5-difluorobenzoic acid (1 g, 4.2 mmol) in dry THF (10 mL), under a nitrogen atmosphere, BH₃·SMe₂ (2 equiv., 8.4 mmol, 4.2 mL, 2 M THF) was added. The reaction mixture was stirred for 17 h at 25° C., cooled down to 0° C. and quenched by the addition of a saturated aqueous Na₂CO₃ solution. The aqueous layer was extracted three times with EtOAc and the combined organic layers were washed once with brine, dried over MgSO₄ and concentrated under reduced pressure to afford the desired product as a brown solid (789 mg, 83% yield).

To a stirred solution of the corresponding alcohol (789 mg, 3.54 mmol) in DCM (10 mL), SOCl 2 (1.5 equiv., 5.3 mmol, 385 μL) and DMF (1 drop) were added. The reaction mixture was stirred at 25° C. for 3 h and concentrated under reduced pressure to afford the desired chloroalkane, which was engaged in the next step without further purification. To a stirred solution of the corresponding chloroalkane (425 mg, 1.76 mmol) in dimethylformamide (5 mL), 4,4-dimethylpiperidine hydrochloride (1 equiv., 1.76 mmol, 263 mg) and K₂CO₃ (2 equiv. 3.52 mmol, 486 mg) were added. The reaction mixture was stirred at 25° C. for 3 days and concentrated under reduced pressure. The obtained residue was purified by flash column chromatography (EtOAc/heptane=3:100 to 10:100) to afford the desired product as a colorless liquid (514 mg, 92%). LRMS (ESI) m/z calcd for [C₁₄H₁₉BrF₂N]⁺: 318.1 found: 318.1

8-(4-((4,4-Dimethylpiperidin-1-yl)methyl)phenyl)-2-(6-(methylamino)pyrimidin-4-yl)-2,5,8-triazaspiro[3.5]nonan-6-one (11):

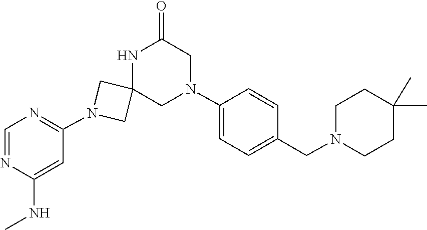

The corresponding Boc protected amine was obtained following the general procedure for Buchwald-Hartwig coupling (chromatography: DCM/MeOH=100:5 to 100:8 to 100:11 to 100:15). The impure desired product was engaged in the next step without further purification.

The corresponding amine was obtained following the general procedure for Boc group deprotection. The impure desired product was engaged in the next step without further purification.

The corresponding chloropyrimidine was obtained following the general procedure for S_{N}Ar with 4,6-dichloropyrimidine. Due to 40 derivative impurities still present, 7 equivalents of the pyrimidine and 7 equivalents of Et₃N were used and the reaction was heated for 7 h at 80° C. in the microwave. The impure desired product was engaged in the next step without further purification.

Compound 11 was obtained following the general procedure for S_{N}Ar with chloropyrimidine derivatives (chromatography: DCM/MeOH=100:0 to 100:10 in 15 min, 100:10 for 5 min, 100:10 to 100:13 in 5 min, 100:13 for 5 min). Yellow solid, 19% yield over four steps. Mp: Decomposition; HRMS (ESI): m/z: calcd for [C₂₅ H₃₆ N₇O]⁺: 450.2981 found: 450.2976.

tert-Butyl 3-amino-3-(nitromethyl)azetidine-1-carboxylate (38):

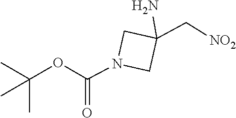 [SMILES: CC(C)(C)OC(=O)N1CC(N)(C[N+](=O)[O-])C1]

To a stirred solution of tert-butyl 3-oxoazetidine-1-carboxylate (10.65 g, 62 mmol) in EtOH (31 mL), MeNO₂ (13 mL) and K₂CO₃ (1 mol %, 0.62 mmol, 86 mg) were added. The reaction mixture was stirred at 25° C. for 17 h and filtered. The filtrate was concentrated under reduced pressure to afford the desired product, which was engaged in the next step without further purification.

To a stirred solution of the corresponding alcohol (62 mmol) in dry DCM (250 mL), under a nitrogen atmosphere and cooled to −78° C., DAST (1.2 eq., 74.4 mmol, 9.8 mL) was added dropwise. The cooling bath was removed and the reaction mixture was stirred for 3 h, cooled to 0° C. and quenched slowly by the addition of a saturated aqueous NaHCO₃ solution. The aqueous layer was extracted three times with DCM, washed once with brine, dried over MgSO₄, filtered and concentrated under reduced pressure to obtain the desired product, which was engaged in the next step without further purification.

The corresponding nitromethylene (62 mmol) was dissolved in ammonia (17.7 mL, 7 N in MeOH) and the reaction mixture was stirred for 2 h at 25° C. The reaction mixture was concentrated under reduced pressure to afford the desired product as an orange solid (15.67 g, quantitative yield over three steps).

tert-Butyl 3-(aminomethyl)-3-(((benzyloxy)carbonyl)amino)azetidine-1-carboxylate (39):

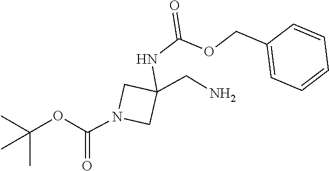 [SMILES: CC(C)(C)OC(=O)N1CC(CN)(NC(=O)OCc2ccccc2)C1]

To a stirred solution of 38 (62 mmol) in dichloromethane (100 mL), a solution of NaHCO₃ (2 equiv., 124 mmol, 10.42 g) in water (100 mL) was added. The reaction mixture was cooled to 0° C. and CbzCl (1 equiv., 62 mmol, 8.8 mL) was added dropwise. The reaction mixture was stirred at 25° C. for 17 h and the two phases were separated. The aqueous layer was extracted two times with DCM. The combined organic layers were washed once with brine, dried over MgSO₄, filtered and concentrated reduced pressure to afford the desired product, which was engaged in the next step without further purification.

To a stirred solution of the corresponding nitroalkane (62 mmol) in dry MeOH (300 mL), under a nitrogen atmosphere at 0° C., NiCl₂·6 H₂O (1 equiv., 62 mmol, 16.9 g) was added, followed by NaBH₄ (5 equiv., 310 mmol, 11.7 g) portionwise to avoid strong H₂ evolution. Caution when adding NaBH₄, the reaction is highly exothermic and produce hydrogen gas. The reaction mixture was stirred at 25° C. for 1 h and quenched by adding saturated aqueous NaHCO₃ solution. The mixture was filtered through a pad of Celite, the filtrate was concentrated under reduced pressure and the obtained residue was diluted with brine and a saturated aqueous Na₂CO₃ solution. The aqueous layer was extracted three times with DCM and the combined organic layers were washed once with brine, dried over MgSO₄, filtered and concentrated under reduced pressure. The crude residue was purified by flash column chromatography (DCM/MeOH/NH₄OH=100:3:0 to 100:3:1 to 100:5:1 to 100:8:1 to 100:12:1 to 100:20:1) to afford the desired product as a white solid (11.3 g, 54% yield over two steps). LRMS (ESI) m/z calcd for [C₃₄ H₅₁N₆O₈]⁺=[2 M+H]⁺: 671.4 found: 671.4.

tert-Butyl 6-oxo-2,5,8-triazaspiro[3.5]nonane-2-carboxylate (40):

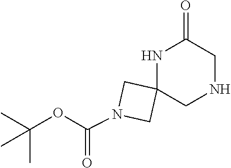 [SMILES: CC(C)(C)OC(=O)N1CC2(CNCC(=O)N2)C1]

To a stirred solution of 39 (11.3 g, 33.7 mmol) in DCM (110 mL) at 0° C., Et₃N (1 equiv., 33.7 mmol, 4.7 mL) and ethyl 2-bromoacetate (1 equiv., 33.7 mmol, 3.7 mL) were added. The reaction mixture was stirred at 25° C. for 17 h and diluted with a saturated aqueous NaHCO₃ solution. The aqueous layer was extracted three times with DCM and the combined organic layers were washed once with water, once with brine, dried over MgSO₄, filtered and concentrated under reduced pressure, to afford the impure desired product (12.4 g, 29 mmol), which was engaged in the next step without further purification.

To a stirred solution of the corresponding Cbz protected amine (29 mmol) in iPrOH (240 mL), Pd/C (5 mol 1.5 mmol, 1.6 g, 10% wt) and ammonium formate (6 equiv., 174 mmol, 11 g) were added portionwise. The reaction mixture was stirred at 80° C. for 4 h, cooled to 25° C., filtered through a pad of Celite and concentrated under reduced pressure. The obtained residue was partitioned between DCM and water, the two phases were separated and the aqueous layer was extracted three times with DCM. The combined organic layers were washed once with water, once with brine, dried over MgSO₄, filtered and concentrated under reduced pressure. The crude residue was purified by flash column chromatography (DCM/MeOH=100:5 to 100:8 to 100:10 to 100:15 to 100:20) to afford the desired product as a white solid (2.6 g, 32% yield over two steps). LRMS (ESI) m/z calcd for [C₇H₁₂N₃O₃]⁺=[M-tBu+2 H]⁺: 186.1 found: 186.2

4-(4-((4-Fluoro-4-methylpiperidin-1-yl)methyl)phenyl)-9-(6-(methylamino)pyrimidin-4-yl)-1,4,9-triazaspiro[5.5]undecan-2-one (53):

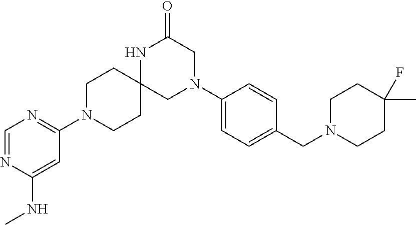

Compound 53 was obtained following the general procedure for S_{N}Ar with chloropyrimidine derivatives (chromatography: DCM/MeOH=100:0 to 100:7 in 20 min, 100:7 for 5 min, 100:7 to 100:8 in 5 min, 100:8 for 10 min). Pale yellow solid, 53% yield. Mp: 203-205° C.; HRMS (ESI): m/z: calcd for [C₂₆ H₃₇ FN₇ O]⁺: 482.3044 found: 482.3038.

9-(6-Chloropyrimidin-4-yl)-4-(4-((4-fluoro-4-methylpiperidin-1-yl)methyl)phenyl)-1,4,9-triazaspiro[5.5]undecan-2-one (54)

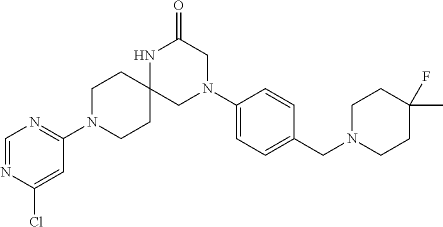 [SMILES: CC1(F)CCN(Cc2ccc(N3CC(=O)NC4(CCN(c5cc(Cl)ncn5)CC4)C3)cc2)CC1]

The corresponding Boc protected amine was obtained following the general procedure for Buchwald-Hartwig coupling (chromatography: EtOAc/heptane=7:3 to 9:1 to EtOAc/MeOH=100:3 to 100:5 to 100:8 to 100:10). The impure desired product was engaged in the next step without further purification.

The corresponding amine was obtained following the general procedure for Boc group deprotection. The impure desired product was engaged in the next step without further purification.

Intermediate 54 was obtained following the general procedure for S_{N}Ar with 4,6-dichloropyrimidine (column chromatography: DCM/MeOH=100:3 to 100:5 to 100.8) to afford the desired product as a white solid (12% yield over three steps).

1-(4-Bromobenzyl)-4-fluoro-4-methylpiperidine (55):

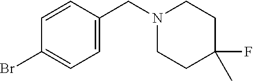 [SMILES: CC1(F)CCN(Cc2ccc(Br)cc2)CC1]

To a stirred solution of tert-butyl 4-hydroxy-4-methylpiperidine-1-carboxylate (500 mg, 2.32 mmol) in dry DCM (7 mL), at 0° C. under a nitrogen atmosphere, DAST (1.5 eq., 3.48 mmol, 460 μL) was added. The mixture was stirred at 25° C. for 3 h and quenched by adding saturated aqueous NaHCO₃ solution. The two phases were separated and the aqueous layer was extracted two times with DCM. The combined organic layers were washed with brine, dried over MgSO₄, filtered and concentrated under reduced pressure to afford the desired product, which was engaged in the next step without further purification.

The corresponding amine was obtained following the general procedure for Boc group deprotection. The impure desired product was engaged in the next step without further purification.

Intermediate 55 was obtained following the general procedure for dimethylpiperidine alkylation. (column chromatography: EtOAc/heptane=1:9 to 3:7 to 1:1). Yellow oil, 77% yield over three steps.

4-(4-((4,4-dimethylpiperidin-1-yl)methyl)phenyl)-9-(1H-pyrazolo[3,4-d]pyrimidin-4-yl)-1,4,9-triazaspiro[5.5]undecan-2-one (56):

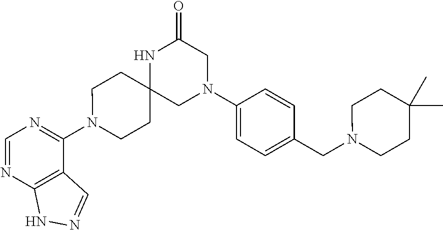 [SMILES: CC1(C)CCN(Cc2ccc(N3CC(=O)NC4(CCN(c5ncnc6[nH]ncc56)CC4)C3)cc2)CC1]

To a stirred solution of 36 (150 mg, 0.31 mmol) in iPrOH (1.5 mL), 2,4-dichloro-7H-pyrrolo[2,3-d]pyrimidine (1.2 equiv., 0.38 mmol, 58 mg) and Et₃N (4 equiv., 1.24 mmol, 172 μL) were added. The reaction mixture was stirred at 50° C. for 3 h and an additional 2 h at 70° C., both in the microwave. The reaction mixture was concentrated under reduced pressure and the crude residue was triturated in water. The resulting precipitate was filtered, washed with water, washed with DCM and dried to afford the desired impure product, which was further purified by flash column chromatography (DCM/MeOH=100:0 to 100:10 in 20 min, 100:10 for 10 min) to afford the desired product as a yellow solid (33 mg, 22% yield). Mp: 252-254° C.; HRMS (ESI): m/z: calcd for [C₂₇H₃₇N₈O]⁺: 489.3090 found: 489.3085

TABLE 1
IC50 data for N6-adenosine-methyltransferase

| Name | Structure | IC₅₀ (μM) |
|---|---|---|
| 53 | 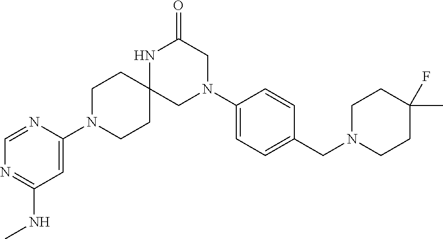 [SMILES: CNc1cc(N2CCC3(CC2)CN(c2ccc(CN4CCC(C)(F)CC4)cc2)CC(=O)N3)ncn1] | 0.069 |
| 7 | 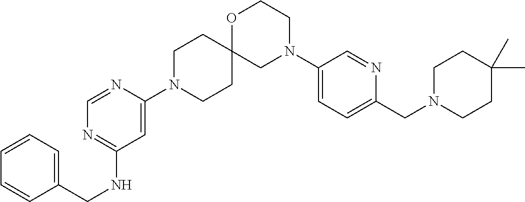 [SMILES: CC1(C)CCN(Cc2ccc(N3CCOC4(CCN(c5cc(NCc6ccccc6)ncn5)CC4)C3)cn2)CC1] | 0.28 |
| 8 | 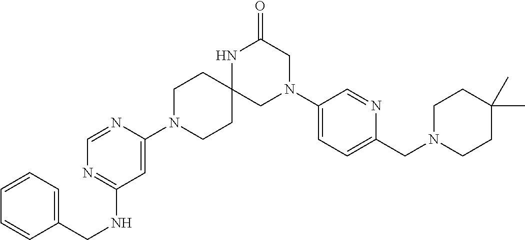 [SMILES: CC1(C)CCN(Cc2ccc(N3CC(=O)NC4(CCN(c5cc(NCc6ccccc6)ncn5)CC4)C3)cn2)CC1] | 0.037 |
| 9 | 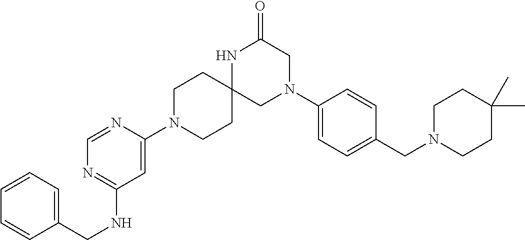 [SMILES: CC1(C)CCN(Cc2ccc(N3CC(=O)NC4(CCN(c5cc(NCc6ccccc6)ncn5)CC4)C3)cc2)CC1] | 0.026 |
| 10 | 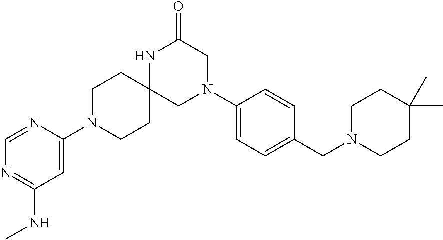 [SMILES: CNc1cc(N2CCC3(CC2)CN(c2ccc(CN4CCC(C)(C)CC4)cc2)CC(=O)N3)ncn1] | 0.089 |
| 11 | 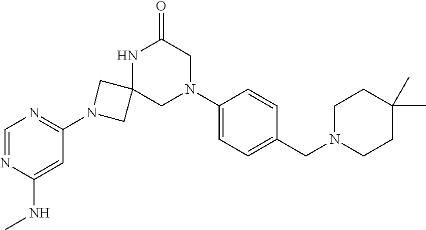 [SMILES: CNc1cc(N2CC3(CN(c4ccc(CN5CCC(C)(C)CC5)cc4)CC(=O)N3)C2)ncn1] | 0.44 |
| 17 | 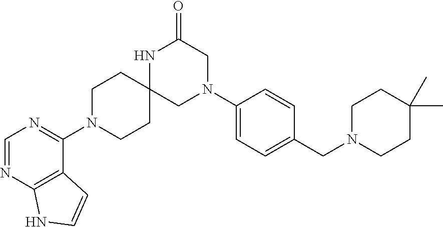 [SMILES: CC1(C)CCN(Cc2ccc(N3CC(=O)NC4(CCN(c5ncnc6[nH]ccc56)CC4)C3)cc2)CC1] | 0.061 |
| 56 | 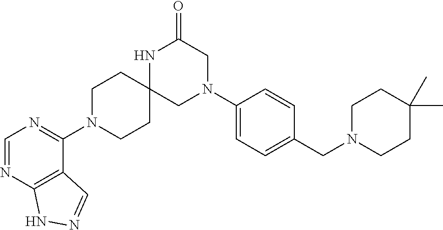 [SMILES: CC1(C)CCN(Cc2ccc(N3CC(=O)NC4(CCN(c5ncnc6[nH]ncc56)CC4)C3)cc2)CC1] | 0.14 |
| 19 | 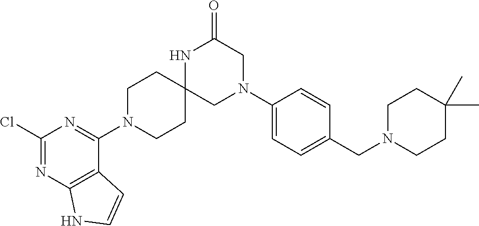 [SMILES: CC1(C)CCN(Cc2ccc(N3CC(=O)NC4(CCN(c5nc(Cl)nc6[nH]ccc56)CC4)C3)cc2)CC1] | 0.024 |
| 16 | 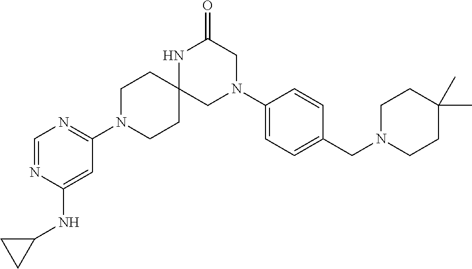 [SMILES: CC1(C)CCN(Cc2ccc(N3CC(=O)NC4(CCN(c5cc(NC6CC6)ncn5)CC4)C3)cc2)CC1] | 0.084 |
| 15 | 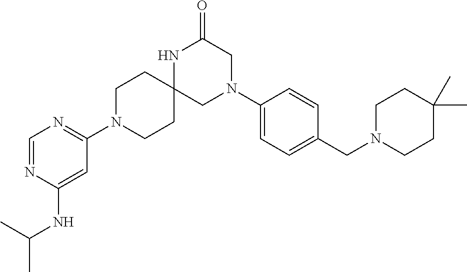 [SMILES: CC(C)Nc1cc(N2CCC3(CC2)CN(c2ccc(CN4CCC(C)(C)CC4)cc2)CC(=O)N3)ncn1] | 0.33 |
| 20 | 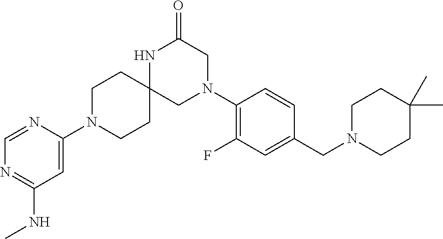 [SMILES: CNc1cc(N2CCC3(CC2)CN(c2ccc(CN4CCC(C)(C)CC4)cc2F)CC(=O)N3)ncn1] | 0.038 |
| 21 | 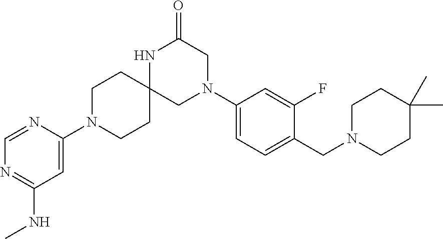 [SMILES: CNc1cc(N2CCC3(CC2)CN(c2ccc(CN4CCC(C)(C)CC4)c(F)c2)CC(=O)N3)ncn1] | 0.032 |
| 22 (UZH2) | 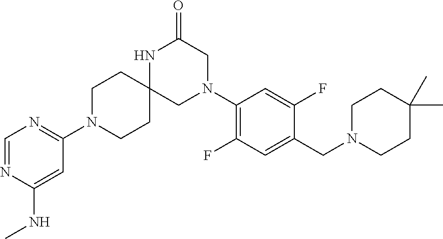 [SMILES: CNc1cc(N2CCC3(CC2)CN(c2cc(F)c(CN4CCC(C)(C)CC4)cc2F)CC(=O)N3)ncn1] | 0.008 |

1. A compound of the general formula (A) 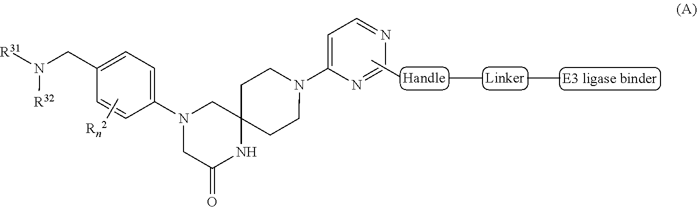
wherein
NR³¹R³² is selected from 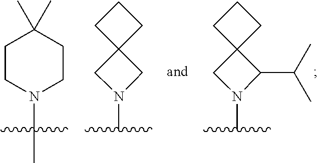
each R² is independently selected from the group comprising F, Cl, CF₃, CHF₂, CH₂ F, particularly each R² is F;
n is an integer selected from 0, 1, 2, 3, and 4, particularly n is an integer selected from 0, 1, and 2, more particularly n is 2;
Handle is a connecting moiety comprising or essentially consisting of 3 to 10 atoms of atomic mass (C, N, O, S), particularly 4 to 8 atoms of atomic mass 12;
Linker is a linker moiety comprising or essentially consisting of 3 to 50 atoms of atomic mass≥12, particularly 4 to 30 atoms of atomic mass≥12, more particularly 5 to 20 atoms of atomic mass≥12;
E3 ligase binder is a moiety specifically binding to an E3 ligase.

2. The compound according to claim 1, wherein the E3 ligase binder is of the formula (B) 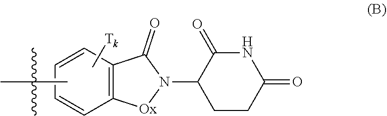
wherein
Ox is CH₂ or C═O;
T is selected from the group comprising F, Cl, particularly T is F;
k is an integer selected from the group comprising 0, 1, 2, particularly the group comprising 0, 1, more particularly k is 0; [symbol]
designates the bond to the Linker.

3. The compound according to claim 2, wherein k is 0.

4. The compound according to any one of the preceding claims, wherein the Handle comprises or essentially consists of 1, 2, 3, or 4 chemical moieties selected from the group comprising alkyl, amine, phenyl, and carbonyl.

5. The compound according to any one of the preceding claims, wherein the Handle is selected from the group comprising the following formulas: 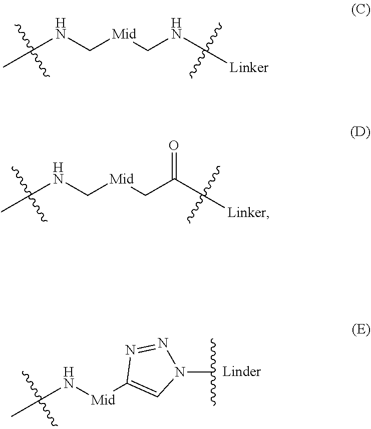
wherein
Mid is selected from the group comprising C₁-C₃ alkyl, and phenyl.

6. The compound according to any one of the preceding claims, wherein the Handle is selected from the group comprising the following formulas:

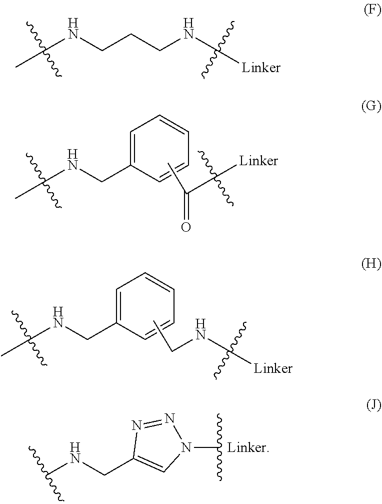

7. The compound according to any one of the preceding claims, wherein the Linker comprises or essentially consists of 1, 2, 3, 4, 5, 6, or 7 chemical moieties independently selected from the group comprising alkyl, aryl, heteroaryl, cycloalkyl, heterocycloalkyl, alkylene, alkylyne, ethylene glycol, carbonyl, ether, ester, amine, amide, sulfonamide, wherein the chemical moieties are each independently unsubstituted or substituted with C₁-C₃ alkyl, halogen, CN, NO₂, hydroxyl, amine, sulfate, phosphate, and/or carboxyl;
particularly wherein the Linker comprises or essentially consists of 1, 2, 3, or 4 chemical moieties selected from the group comprising alkyl, ethylene glycol, carbonyl, piperazine, aryl, amine, triazole.

8. The compound according to any one of the preceding claims, wherein the Linker is selected from the group comprising the following formulas: 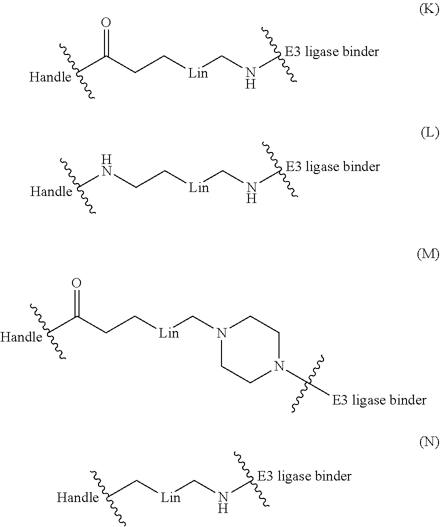
wherein
Lin is selected from the group comprising C₃-C₂₀ alkyl, C₃-C₂₀ alkyl-triazole, oligo(ethylene glycol).

9. The compound according to any one of the preceding claims, wherein the Linker is selected from the group comprising the following formulas: 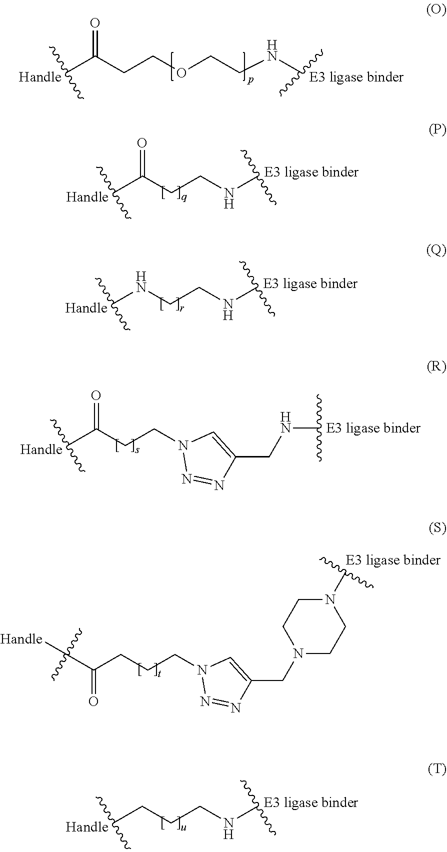
wherein
p is selected from 2, 3, 4, 5;
q is selected from 7, 8, 9, 10, 11, 12, 13;
r is selected from 11, 12, 13, 14, 15, 16, 17;
s is selected from 7, 8, 9, 10, 11, 12, 13;
t is selected from 3, 4, 5, 6, 7, 8, 9;
u is selected from 7, 8, 9, 10, 11, 12, 13.

10. The compound according to any one of the preceding claims 1 to 6, wherein the Linker is a peptide, particularly a peptide consisting of proteinogenic amino acids.

11. The compound according to any one of the preceding claims, wherein
the E3 ligase binder is of the formula (B) as defined in claim 2; and
the Handle has the definition of claim 6; and
the Linker has the definition of claim 9.

12. The compound according to any one of the preceding claims, wherein the compound comprises the following definitions of the Handle, Linker and E3 ligase binder:

| Handle | Linker | E3 ligase binder |
|---|---|---|
| formula (F) | formula (O) | 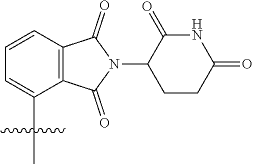 [SMILES: *c1cccc2c1C(=O)N(C1CCC(=O)NC1=O)C2=O] |
| formula (F) | formula (R) | 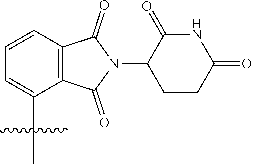 [SMILES: *c1cccc2c1C(=O)N(C1CCC(=O)NC1=O)C2=O] |
| | | 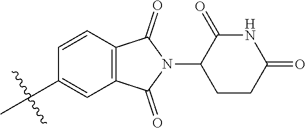 [SMILES: *c1ccc2c(c1)C(=O)N(C1CCC(=O)NC1=O)C2=O] |
| formula (F) | formula (P) | 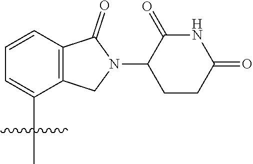 [SMILES: *c1cccc2c1CN(C1CCC(=O)NC1=O)C2=O] |
| | | 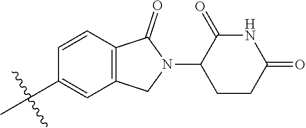 [SMILES: *c1ccc2c(c1)CN(C1CCC(=O)NC1=O)C2=O] |
| formula (F) | formula (S) | 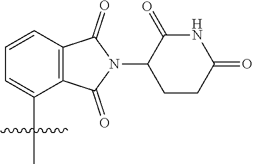 [SMILES: *c1cccc2c1C(=O)N(C1CCC(=O)NC1=O)C2=O] |
| | | 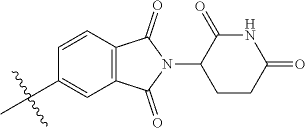 [SMILES: *c1ccc2c(c1)C(=O)N(C1CCC(=O)NC1=O)C2=O] |
| formula (H) | formula (P) | 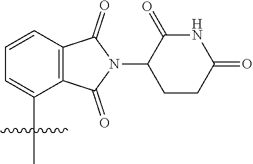 [SMILES: *c1cccc2c1C(=O)N(C1CCC(=O)NC1=O)C2=O] |
| | | 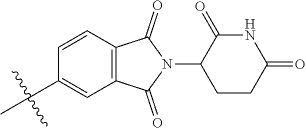 [SMILES: *c1ccc2c(c1)C(=O)N(C1CCC(=O)NC1=O)C2=O] |
| formula (G) | formula (Q) | 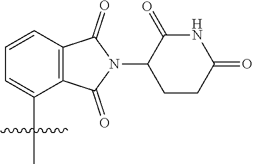 [SMILES: *c1cccc2c1C(=O)N(C1CCC(=O)NC1=O)C2=O] |
| | | 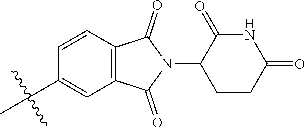 [SMILES: *c1ccc2c(c1)C(=O)N(C1CCC(=O)NC1=O)C2=O] |
| formula (J) | formula (T) | 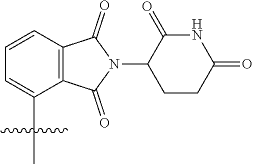 [SMILES: *c1cccc2c1C(=O)N(C1CCC(=O)NC1=O)C2=O] |
| | | 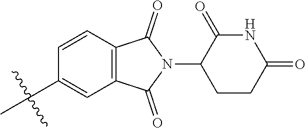 [SMILES: *c1ccc2c(c1)C(=O)N(C1CCC(=O)NC1=O)C2=O] |

13. The compound according to any one of the preceding claims 1 to 12, wherein NR³¹R³² is

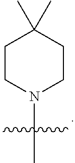 [SMILES: *N1CCC(C)(C)CC1]

14. The compound according to any one of the preceding claims 1 to 12, wherein NR³¹R³² is

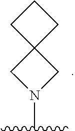 [SMILES: *N1CC2(CCC2)C1]

15. The compound according to any one of the preceding claims 1 to 12, wherein NR³¹R³² is 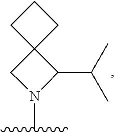 [SMILES: *N1CC2(CCC2)C1C(C)C]
particularly NR³¹R³² is

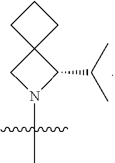 [SMILES: *N1CC2(CCC2)[C@@H]1C(C)C]

16. A compound of the general formula (U) 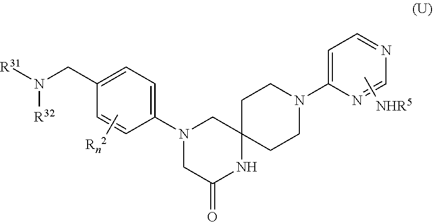
NR³¹R³² is selected from 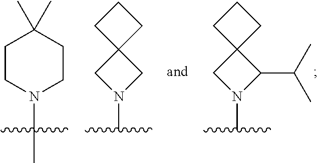
R² is selected from the group comprising F, Cl, CF₃, CHF 2, CH₂ F, particularly R² is F;
n is an integer selected from 0, 1, 2, 3, and 4, particularly n is an integer selected from 0, 1, and 2, more particularly n is 2;
R⁵ is selected from an alkyl, an alkylaryl, a heteroalkylaryl, a cycloalkyl, an aryl, a heteroaryl and a heterocycle, particularly R⁵ is selected from an alkyl, an alkylaryl, and a cycloalkyl, more particularly R⁵ is selected from methyl and methylphenyl.

17. The compound according to claim 16, wherein NR³¹R³² is

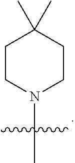 [SMILES: *N1CCC(C)(C)CC1]

18. The compound according to claim 16, wherein NR³¹R³² is

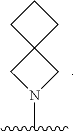 [SMILES: *N1CC2(CCC2)C1]

19. The compound according to claim 16, wherein NR³¹R³² is 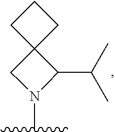 [SMILES: *N1CC2(CCC2)C1C(C)C]
particularly NR³¹R³² is

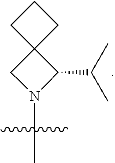 [SMILES: *N1CC2(CCC2)[C@@H]1C(C)C]

20. A compound according to any of the preceding claims for use as a medicament.

21. A compound according to any of the preceding claims 1 to 19 for use in treatment of cancer.

22. The compound for use according to claim 21, wherein said cancer is selected from the group comprising renal cancer, breast cancer, acute myeloid leukemia, hepatocellular carcinoma, and lung adenocarcinoma.
